(19) United States
(12) Patent Application Publication
(10) Pub. No.: US 2022/0298502 A1
(43) Pub. Date: Sep. 22, 2022

(54) NUCLEASE AND NICKASE FUSION PROTEINS FOR INCREASED HOMOLOGOUS RECOMBINATION IN MAMMALIAN CELLS

(71) Applicant: Inscripta, Inc., Boulder, CO (US)

(72) Inventor: Brian Chaikind, Boulder, CO (US)

(21) Appl. No.: 17/696,131

(22) Filed: Mar. 16, 2022

Related U.S. Application Data
(60) Provisional application No. 63/162,158, filed on Mar. 17, 2021.

(51) Int. Cl. C12N 15/10; C12N 9/22

(52) U.S. Cl. CPC C12N 15/102; C12N 9/22; C12N 2310/20; C07K 2319/80; C12N 2800/80

(57) ABSTRACT

The present disclosure provides compositions and methods to increase the percentage of edited cells in a cell population when employing nucleic-acid guided editing, as well as automated instruments for performing these methods.

### RELATED CASES

This application claims priority to U.S. Ser. No. 63/162,158, filed 17 Mar. 2021, entitled “NUCLEASE AND NICKASE FUSION PROTEINS FOR INCREASED HOMOLOGOUS RECOMBINATION IN MAMMALIAN CELLS”, which is incorporated herein in its entirety.

### FIELD OF THE INVENTION

The present disclosure relates to compositions of matter, methods and instruments for editing live cells, particularly mammalian cells, with nuclease or nickase fusion proteins.

### BACKGROUND OF THE INVENTION

In the following discussion certain articles and methods will be described for background and introductory purposes. Nothing contained herein is to be construed as an “admission” of prior art. Applicant expressly reserves the right to demonstrate, where appropriate, that the articles and methods referenced herein do not constitute prior art under the applicable statutory provisions.

The ability to make precise, targeted changes to the genome of living cells has been a long-standing goal in biomedical research and development. Recently various nucleases have been identified that allow manipulation of gene sequence in live cells, and hence gene function. The nucleases include nucleic acid-guided nucleases, which enable researchers to generate permanent edits in live cells. Of course, it is desirable to attain the highest editing rates possible in a cell population: however, in many instances the percentage of edited cells resulting from nucleic acid-guided nuclease editing can be in the single digits or even less than 1%, particularly in mammalian cells.

There is thus a need in the art of nucleic acid-guided nuclease editing for improved methods, compositions, modules and instruments for increasing the efficiency of editing. The present disclosure addresses this need.

### SUMMARY OF THE INVENTION

This Summary is provided to introduce a selection of concepts in a simplified form that are further described below in the Detailed Description. This Summary is not intended to identify key or essential features of the claimed subject matter, nor is it intended to be used to limit the scope of the claimed subject matter. Other features, details, utilities, and advantages of the claimed subject matter will be apparent from the following written Detailed Description including those aspects illustrated in the accompanying drawings and defined in the appended claims.

The present disclosure relates to methods, compositions, and automated cell processing instruments that increase the efficiency of nucleic acid-guided editing in a live cell population using a nucleic acid nuclease fusion protein or a nucleic acid nickase fusion protein, where the nuclease or nickase is fused to a bacterial Ku protein or a bacterial Ku protein monomer, a protein that binds to double-stranded DNA ends, or to a ligase, an enzyme that joins DNA strands together. That is, the present compositions and methods enhance editing efficiency by retaining certain characteristics of nucleic acid-directed nucleases—the binding specificity and ability to cleave one or both DNA strands in a targeted manner—combined the enzymatic CNA ligase activity of a ligase or the DNA binding and DNA repair binding and/or recruitment of a Ku or ligase protein.

Thus, there is provided a system for RNA-guided (CRISPR) editing of live mammalian cells comprising: (a) a fusion protein comprising two domains: (i) an N-terminal or C-terminal domain comprising an RNA-guided nuclease or RNA-guided nickase domain; and (ii) a C-terminal or N-terminal domain comprising a ligase or bacterial Ku protein, wherein the RNA-guided nuclease or RNA-guided nickase and ligase or bacterial Ku protein are separated by a linker; and (b) a gRNA and repair template pair. In some aspects, the RNA-guided nuclease is a nickase domain, and in some aspects, the RNA-guided nickase comprises a CRISPR nucleic acid-guided nuclease engineered to cut one DNA strand in the target DNA rather than making a double-stranded cut, and the nickase portion is fused to a reverse transcriptase. In specific aspects, the one or more nickases include MAD7 nickase, MAD2001 nickase, MAD2007 nickase, MAD2008 nickase, MAD2009 nickase, MAD2011 nickase, MAD2017 nickase, MAD2019 nickase, MAD297 nickase, MAD298 nickase, MAD299 nickase, or other MAD-series nickases, variants thereof, and/or combinations thereof as described in U.S. Pat. Nos. 10,883,077; 11,053,485; 11,085,030; 11,200,089; 11,193,115; and U.S. Ser. No. 17/463,498. In alternative aspects, the RNA-guided nuclease or nickase domain is a nuclease domain, and in some aspects, the RNA-guided nuclease is selected from Cas 9, Cas 12, MAD2, or MAD7, MAD 2007 or other MADzymes and MADzyme systems (see U.S. Pat. Nos. 10,604,746; 10,655,114; 10,649,754; 10,876,102; 10,833,077; 11,053,485; 10,704,022; 10,745,678; 10,724,021; 10,767,169; 10,870,761; 10,011,849; 10,435,714; 10,626,416; 9,982,279; and 10,337,028; and U.S. Ser. Nos. 16/953,253; 17/374,628; 17/200,074; 17/200,089; 17/200,110; 16/953,233; 17/463,498; 63/134,938; 16/819,896; 17/179,193; and 16/421,783 for sequences and other details related to engineered and naturally-occurring MADzymes).

In some aspects of this embodiment, the N-terminal or C-terminal domain comprises a ligase, and in some aspects, the ligase is selected from Taq ligase, PBCV1 ligase, or ligase D from B. subtilis or M. smegmatis. In alternative aspects, the N-terminal or C-terminal domain comprises a bacterial Ku protein, and in some aspects, the bacterial Ku protein monomer is a Ku protein monomer from B. subtilis or M. smegmatis.

In some aspects, the linker is a flexible linker and in alternative aspects, the linker is a rigid linker.

In some aspects, the N-terminal domain comprises the RNA-guided nuclease or RNA-guided nickase and the C-terminal domain comprises the bacterial Ku protein or ligase. In alternative aspects, the C-terminal domain comprises the RNA-guided nuclease or RNA-guided nickase and the N-terminal domain comprises the bacterial Ku protein or ligase.

In some aspects, the bacterial Ku protein is a bacterial protein monomer.

In yet another embodiment there is provided a method for RNA-guided (CRISPR) editing of live mammalian cells comprising designing a fusion protein comprising two domains: (i) an N-terminal or C-terminal domain comprising an RNA-guided nuclease or RNA-guided nickase domain; and (ii) a C-terminal or N-terminal domain comprising a ligase or bacterial Ku protein, wherein the RNA-guided nuclease or RNA-guided nickase and ligase or bacterial Ku protein are separated by a linker; designing a library of at least two different gRNA and repair template pairs; transforming mammalian cells with the fusion protein and library of at least two different gRNA and repair template pairs; providing conditions to allow the mammalian cells to be edited; and enriching for mammalian cells that have been edited.

In some aspects, the enriching step is performed by fluorescence-activated cell sorting, and in some aspects, the enriching step is performed by magnetic-activated cell sorting or antibiotic selection.

Other embodiments provide a system for RNA-guided (CRISPR) editing of live mammalian cells comprising: an N-terminal or C-terminal domain comprising an RNA-guided nuclease or RNA-guided nickase domain; a C-terminal or N-terminal domain comprising a ligase or bacterial Ku protein, wherein the RNA-guided nuclease or RNA-guided nickase and ligase or bacterial Ku protein are separated by a linker; and a gRNA and repair template pair.

These aspects and other features and advantages of the invention are described below in more detail.

### BRIEF DESCRIPTION OF THE DRAWINGS

The foregoing and other features and advantages of the present invention will be more fully understood from the following detailed description of illustrative embodiments taken in conjunction with the accompanying drawings in which:

FIG. 1 is a simplified block diagram of an exemplary method for editing live cells utilizing either a nucleic acid-guided nuclease-Ku, nuclease-ligase, nickase-Ku or nickase-ligase fusion protein.

FIG. 2A depicts an exemplary workflow employing microcarrier-partitioned delivery for editing cells. FIG. 2B depicts an alternative workflow employing microcarrier-partitioned delivery for editing cells.

FIGS. 3A-3C depict various components of exemplary embodiments of a bioreactor module included in an integrated instrument useful for growing and transfecting cells. FIGS. 3D and 3E depict an exemplary integrated instrument for growing and transfecting cells.

FIG. 4 is a simplified graphic showing two workflows used to test the nuclease and nickase fusion proteins.

FIG. 5 a bar graph demonstrating that the Cas9 nickase H840A fused with a B. subtilis Ku, with and without unfused proteins increases donor DNA mediated repair in HEK293t cells.

FIG. 6 is another simplified graphic showing a workflow used to test the nuclease and nickase fusion proteins.

FIG. 7 is a bar graph showing the levels of editing achieved using the Cas9 nickase H840A fused with various ligases or monomers of Ku proteins from B. subtilis and M. smegmatis in HEK293t cells.

FIG. 8 is a bar graph demonstrating that the Cas9 nickase D10A fused with a B. subtilis Ku protein shows little to no improvement in donor DNA mediated repair in HEK293t cells over Cas 9 nickase D10A alone.

FIG. 9 is a bar graph showing that the addition of unfused variants does not have an effect. However, the Cas9-KuBsub and Cas9-LigDBsub do result in higher editing efficiencies. Additionally, under slightly different transfection regimes Cas9-LigDBsub and Cas9-KuBsub result in up to a two-fold increase in editing relative to Cas9.

FIG. 10 illustrates a workflow for the transfections used for the experiments that generated the results shown in FIGS. 11-15.

FIG. 11 illustrates the differences between the assays used to assess genomic repair. Top assay used in FIGS. 5, 7 8 and 9. Bottom assay are used in FIGS. 12, 13, 14, and 15.

FIG. 12 shows the donor mediated repair results for the GFP→BFP conversion at a nick-to-edit distance of 3.

FIG. 13 indicates error-prone repair of the GFP→BFP conversion at a nick-to-edit distance of 3.

FIG. 14 shows the donor mediated repair results for the GFP→BFP conversion at a nick-to-edit distance of 31.

FIG. 15 indicates error-prone repair of the GFP→BFP conversion at a nick-to-edit distance of 31.

FIG. 16 illustrates how Cas9 H840A nickase or Cas9 WT fusion proteins effect donor-mediated repair using a donor that encodes for a 187 bp insert.

The results shown in FIG. 17 demonstrate co-transfections with a fusion protein, a plasmid encoding a gRNA and a donor enabled repair.

It should be understood that the drawings are not necessarily to scale, and that like reference numbers refer to like features.

### DETAILED DESCRIPTION

All of the functionalities described in connection with one embodiment of the methods, devices or instruments described herein are intended to be applicable to the additional embodiments of the methods, devices and instruments described herein except where expressly stated or where the feature or function is incompatible with the additional embodiments. For example, where a given feature or function is expressly described in connection with one embodiment but not expressly mentioned in connection with an alternative embodiment, it should be understood that the feature or function may be deployed, utilized, or implemented in connection with the alternative embodiment unless the feature or function is incompatible with the alternative embodiment.

The practice of the techniques described herein may employ, unless otherwise indicated, techniques and descriptions of molecular biology (including recombinant techniques), cell biology, biochemistry, and genetic engineering technology, which are within the skill of those who practice in the art given the present disclosure. Such conventional techniques and descriptions can be found in standard laboratory manuals such as Green and Sambrook, Molecular Cloning: A Laboratory Manual, 4th Ed., Cold Spring Harbor Laboratory Press, Cold Spring Harbor, N.Y., (2014); Current Protocols in Molecular Biology, Ausubel, et al. eds., (2017); Neumann, et al., Electroporation and Electrofusion in Cell Biology, Plenum Press, N Y (1989); and Chang, et al., Guide to Electroporation and Electrofusion, Academic Press, C A (1992); Cell and Tissue Culture: Laboratory Procedures in Biotechnology (Doyle & Griffiths, eds., John Wiley & Sons (1998)); Mammalian Chromosome Engineering—Methods and Protocols (G. Hadlaczky, ed., Humana Press (2011)); Essential Stem Cell Methods, (Lanza and Klimanskaya, eds., Academic Press (2011)); Stem Cell Therapies: Opportunities for Ensuring the Quality and Safety of Clinical Offerings: Summary of a Joint Workshop (Board on Health Sciences Policy, National Academies Press (2014)); Essentials of Stem Cell Biology, 3rd Ed., (Lanza and Atala, eds., Academic Press (2013)); and Handbook of Stem Cells (Atala and Lanza, eds., Academic Press (2012)), all of which are herein incorporated in their entirety by reference for all purposes. Nucleic acid-guided nuclease techniques can be found in, e.g., Genome Editing and Engineering from TALENs and CRISPRs to Molecular Surgery, Appasani and Church (2018); and CRISPR: Methods and Protocols, Lindgren and Charpentier (2015); both of which are herein incorporated in their entirety by reference for all purposes.

Note that as used herein and in the appended claims, the singular forms “a,” “an,” and “the” include plural referents unless the context clearly dictates otherwise. Thus, for example, reference to “a cell” refers to one or more cells, and reference to “the system” includes reference to equivalent steps, methods and devices known to those skilled in the art, and so forth. Additionally, it is to be understood that terms such as “left,” “right,” “top,” “bottom,” “front,” “rear,” “side,” “height,” “length,” “width,” “upper,” “lower,” “interior,” “exterior,” “inner,” “outer” that may be used herein merely describe points of reference and do not necessarily limit embodiments of the present disclosure to any particular orientation or configuration. Furthermore, terms such as “first,” “second,” “third,” etc., merely identify one of a number of portions, components, steps, operations, functions, and/or points of reference as disclosed herein, and likewise do not necessarily limit embodiments of the present disclosure to any particular configuration or orientation.

Unless defined otherwise, all technical and scientific terms used herein have the same meaning as commonly understood by one of ordinary skill in the art to which this invention belongs. All publications mentioned herein are incorporated by reference for the purpose of describing and disclosing devices, formulations and methodologies that may be used in connection with the presently described invention.

Where a range of values is provided, it is understood that each intervening value, between the upper and lower limit of that range and any other stated or intervening value in that stated range is encompassed within the invention. The upper and lower limits of these smaller ranges may independently be included in smaller ranges, and are also encompassed within the invention, subject to any specifically excluded limit in the stated range. Where the stated range includes one or both of the limits, ranges excluding either or both of those included limits are also included in the invention.

In the following description, numerous specific details are set forth to provide a more thorough understanding of the present invention. However, it will be apparent to one of skill in the art that the present invention may be practiced without one or more of these specific details. In other instances, features and procedures well known to those skilled in the art have not been described in order to avoid obscuring the invention. The terms used herein are intended to have the plain and ordinary meaning as understood by those of ordinary skill in the art.

The term “complementary” as used herein refers to Watson-Crick base pairing between nucleotides and specifically refers to nucleotides hydrogen bonded to one another with thymine or uracil residues linked to adenine residues by two hydrogen bonds and cytosine and guanine residues linked by three hydrogen bonds. In general, a nucleic acid includes a nucleotide sequence described as having a “percent complementarity” or “percent homology” to a specified second nucleotide sequence. For example, a nucleotide sequence may have 80%, 90%, or 100% complementarity to a specified second nucleotide sequence, indicating that 8 of 10, 9 of 10 or 10 of 10 nucleotides of a sequence are complementary to the specified second nucleotide sequence. For instance, the nucleotide sequence 3′-TCGA-5′ is 100% complementary to the nucleotide sequence 5′-AGCT-3′; and the nucleotide sequence 3′-TCGA-5′ is 100% complementary to a region of the nucleotide sequence 5′-TAGCTG-3′.

The term DNA “control sequences” refers collectively to promoter sequences, polyadenylation signals, transcription termination sequences, upstream regulatory domains, origins of replication, internal ribosome entry sites, nuclear localization sequences, enhancers, and the like, which collectively provide for the replication, transcription and translation of a coding sequence in a recipient cell. Not all of these types of control sequences need to be present so long as a selected sequence is capable of being replicated, transcribed and—for some components—translated in an appropriate host cell.

The term “editing cassette” refers to a nucleic acid molecule comprising a coding sequence for transcription of a guide nucleic acid or gRNA covalently linked to a coding sequence for transcription of a repair template.

The terms “enrich” or “enrichment” refer to 1) identifying cells that have received the editing components needed to perform the intended editing operation; or 2) identifying edited cells. Enrichment can be performed directly or by using surrogates, e.g., cell surface handles co-introduced with one or more of the editing components. Enrichment includes physical enrichment of cells expressing a selectable marker, including fluorescent-activated cell sorting selection, magnetic-activated cell sorting selection, antibiotic selection, and the like.

The terms “guide nucleic acid” or “guide RNA” or “gRNA” refer to a polynucleotide comprising 1) a guide sequence capable of hybridizing to a genomic target locus, and 2) a scaffold sequence capable of interacting or complexing with a nucleic acid-guided nuclease or nickase fusion.

“Homology” or “identity” or “similarity” refer to sequence similarity between two peptides or, more often in the context of the present disclosure, between two nucleic acid molecules. Homology can be determined by comparing a position in each sequence which may be aligned for purposes of comparison. When a position in the compared sequence is occupied by the same base or amino acid, then the molecules are homologous at that position. A degree of homology between sequences is a function of the number of matching or homologous positions shared by the sequences.

As used herein, the terms “nucleic acid-guided nickase fusion”, “nickase fusion enzyme”, or “nickase fusion” refers to a nucleic acid-guided nickase—or nucleic acid-guided nuclease or CRISPR nuclease that has been engineered to act as a nickase rather than a nuclease that initiates double-stranded DNA breaks—where the nucleic acid-guided nickase nicks only one strand of a double-strand DNA target sequence (or is engineered to nick one strand quickly and the other strand more slowly after the first strand is nicked) and is fused to a Ku protein or fused to a ligase enzyme.

“Operably linked” refers to an arrangement of elements where the components so described are configured so as to perform their usual function. Thus, control sequences operably linked to a coding sequence are capable of effecting the transcription of a sequence, and in some cases the translation of a coding sequence. The control sequences need not be contiguous with the coding sequence so long as they function to direct the expression of the coding sequence. Thus, for example, intervening untranslated yet transcribed sequences can be present between a promoter sequence and the coding sequence and the promoter sequence can still be considered “operably linked” to the coding sequence. In fact, such sequences need not reside on the same contiguous DNA molecule (i.e. chromosome) and may still have interactions resulting in altered regulation.

A “PAM (protospacer adjacent motif) mutation” refers to one or more edits to a target sequence that removes, mutates, or otherwise renders inactive a PAM (protospacer adjacent motif) or spacer region in the target sequence.

As used herein, the terms “protein” and “polypeptide” are used interchangeably. Proteins may or may not be made up entirely of amino acids.

A “promoter” or “promoter sequence” is a DNA regulatory region capable of binding RNA polymerase and initiating transcription of a polynucleotide or polypeptide coding sequence such as messenger RNA, ribosomal RNA, small nuclear or nucleolar RNA, guide RNA, or any kind of RNA transcribed by any class of any RNA polymerase I, II or III. Promoters may be constitutive or inducible.

As used herein the term “repair template” or “donor template” refers to nucleic acid that is designed to introduce a DNA sequence modification (insertion, deletion, substitution) into a locus by homologous recombination or another donor-mediated repair using a nucleic acid-guided nuclease or a nucleic acid that serves as a template (including a desired edit) to be incorporated into target DNA by reverse transcriptase using a nickase fusion enzyme.

As used herein the term “selectable marker” refers to a gene introduced into a cell, which confers a trait suitable for artificial selection. General use selectable markers are well-known to those of ordinary skill in the art. Drug selectable markers such as ampicillin/carbenicillin, kanamycin, nourseothricin N-acetyl transferase, chloramphenicol, erythromycin, tetracycline, gentamicin, bleomycin, streptomycin, rifampicin, puromycin, hygromycin, blasticidin, and G418 may be employed. In other embodiments, selectable markers include, but are not limited to sugars such as rhamnose, human nerve growth factor receptor (detected with a MAb, such as described in U.S. Pat. No. 6,365,373); truncated human growth factor receptor (detected with MAb); mutant human dihydrofolate reductase (DHFR; fluorescent MTX substrate available); secreted alkaline phosphatase (SEAP; fluorescent substrate available); human thymidylate synthase (TS; confers resistance to anti-cancer agent fluorodeoxyuridine); human glutathione S-transferase alpha (GSTA1; conjugates glutathione to the stem cell selective alkylator busulfan; chemoprotective selectable marker in CD34+ cells); CD24 cell surface antigen in hematopoietic stem cells; human CAD gene to confer resistance to N-phosphonacetyl-L-aspartate (PALA); human multi-drug resistance-1 (MDR-1; P-glycoprotein surface protein selectable by increased drug resistance or enriched by FACS); human CD25 (IL-2a; detectable by Mab-FITC); Methylguanine-DNA methyltransferase (MGMT; selectable by carmustine); and Cytidine deaminase (CD; selectable by Ara-C). Also useful are fluorescent tags selected from, e.g., GFP, BFP, RFP, TagBFP, TagCFP, TagGFP2, TagYFP, TagRFP, FusionRed, mKate2, TurboGFP, TurboYFP, TurboRFP, TurboFP602, TurboFP635, TurboFP650, AmCyan1, AcvGFP1, ZsGreen1, ZsYellow1, mBanana, mOrange, mOrange2, DsRed-Express2, EsRed-Express, tdTomato, DsRed-Monomer, DsRed2, AsRed2, mStrawberry, mCherry, HcRedl, mRaspberry, E2-Crimson, mPlum, Dendra 2, Timer, and PAmCherry, HALO-tags, or infrared-shifted fluorescent proteins “Selective medium” as used herein refers to cell growth medium to which has been added a chemical compound or biological moiety that selects for or against selectable markers.

The terms “target genomic DNA sequence”, “cellular target sequence”, “target sequence” or “genomic target locus” refer to any locus in vitro or in vivo, or in a nucleic acid (e.g., genome) of a cell or population of cells, in which a change of at least one nucleotide is desired using a nucleic acid-guided nuclease editing system. The cellular target sequence can be a genomic locus or extrachromosomal locus.

The terms “transformation”, “transfection” and “transduction” are used interchangeably herein to refer to the process of introducing exogenous DNA into cells.

The term “variant” may refer to a polypeptide or polynucleotide that differs from a reference polypeptide or polynucleotide but retains essential properties. A typical variant of a polypeptide differs in amino acid sequence from another reference polypeptide. Generally, differences are limited so that the sequences of the reference polypeptide and the variant are closely similar overall and, in many regions, identical. A variant and reference polypeptide may differ in amino acid sequence by one or more modifications (e.g., substitutions, additions, and/or deletions). A variant of a polypeptide may be a conservatively modified variant. A substituted or inserted amino acid residue may or may not be one encoded by the genetic code (e.g., a non-natural amino acid). A variant of a polypeptide may be naturally occurring, such as an allelic variant, or it may be a variant that is not known to occur naturally.

A “vector” is any of a variety of nucleic acids that comprise a desired sequence or sequences to be delivered to and/or expressed in a cell. Vectors are typically composed of DNA, although RNA vectors are also available. Vectors include, but are not limited to, plasmids, fosmids, phagemids, virus genomes, synthetic chromosomes, and the like.

### Nuclease-Directed Genome Editing Generally

The compositions and methods described herein are employed to perform nuclease-directed or nickase fusion-directed genome editing (e.g., RNA-guided nuclease (RGN) or CRISPR editing) to introduce desired edits to a population of live cells. The most commonly employed method for using RGNs to introduce precision edits is to co-deliver an appropriate gRNA and repair template where the repair template is designed to introduce a DNA modification into a locus by leveraging homology-directed repair (HDR) or other innate repair mechanism at the intended site where the repair template is designed to serve as a template (with a desired edit) to be incorporated into the target DNA. In many organisms, however, innate inefficiencies in HDR lead to high levels of toxicity associated with double-strand DNA breaks. For example, cells that receive inactive gRNAs out-compete cells that go through the editing process since cells with inactive gRNAs are not subject to double-strand DNA breaks. Likewise, non-homologous end joining (NHEJ) in mammalian systems outcompete HDR leading to an increase in error-prone repair outcomes, which are an impediment to precision editing. Thus, creating methods and compositions for increasing HDR or template-directed repair is a goal in the art of nucleic acid-guided nuclease editing.

In the nucleic acid-guided nuclease or nickase fusion editing process, a nucleic acid-guided nuclease or nickase fusion complexed with an appropriate synthetic guide nucleic acid (e.g., gRNA) in a cell cuts or nicks the genome of the cell at a desired location. The guide nucleic acid helps the nucleic acid-guided nuclease or nickase fusion recognize and cut the DNA at a specific target sequence. By manipulating the nucleotide sequence of the guide nucleic acid, the nucleic acid-guided nuclease or nickase fusion may be programmed to target any DNA sequence for cleavage as long as an appropriate protospacer adjacent motif (PAM) is nearby. Thus, the gRNA comprises homology to the target sequence. In certain aspects, the nucleic acid-guided nuclease or nickase fusion editing system may use two separate guide nucleic acid molecules that combine to function as a guide nucleic acid, e.g., a CRISPR RNA (crRNA) and trans-activating CRISPR RNA (tracrRNA). In other aspects, the guide nucleic acid is a single guide nucleic acid construct that includes both 1) a guide sequence capable of hybridizing to a genomic target locus, and 2) a scaffold sequence capable of interacting or complexing with a nucleic acid-guided nuclease or nickase fusion.

In general, a guide nucleic acid (e.g., gRNA) complexes with a compatible nucleic acid-guided nuclease or nickase fusion and can then hybridize with a target sequence, thereby directing the nuclease or nickase fusion to the target sequence. A guide nucleic acid can be DNA or RNA; alternatively, a guide nucleic acid may comprise both DNA and RNA. In some embodiments, a guide nucleic acid may comprise modified or non-naturally occurring nucleotides. In cases where the guide nucleic acid comprises RNA, the gRNA may be encoded by a DNA sequence on a polynucleotide molecule such as a plasmid, linear construct, or the coding sequence may and preferably does reside within an editing cassette. Methods and compositions for designing and synthesizing editing cassettes are described in U.S. Pat. Nos. 10,240,167; 10,266,849; 9,982,278; 10,351,877; 10,364,442; 10,435,715; 10,669,559; 10,711,284; 10,731,180, all of which are incorporated by reference herein in their entirety. Editing cassettes, in addition to paired gRNAs and repair templates, may and typically do in the context of the present disclosure comprise additional sequences such as cassette barcodes and/or cassette capture sequences where the capture sequences facilitate capture of the editing cassettes when analyzing the edit/cellular nucleic acid profile relationship.

A guide nucleic acid comprises a guide sequence, where the guide sequence is a polynucleotide sequence having sufficient complementarity with a target sequence to hybridize with the target sequence and direct sequence-specific binding of a complexed nucleic acid-guided nuclease or nickase fusion to the target sequence. The degree of complementarity between a guide sequence and the corresponding target sequence, when optimally aligned using a suitable alignment algorithm, is about or more than about 50%, 60%, 75%, 80%, 85%, 90%, 95%, 97.5%, 99% or more. Optimal alignment may be determined with the use of any suitable algorithm for aligning sequences. In some embodiments, a guide sequence is about or more than about 10, 11, 12, 13, 14, 15, 16, 17, 18, 19, 20, 21, 22, 23, 24, 25, 26, 27, 28, 29, 30, 35, 40, 45, 50, 75, or more nucleotides in length. In some embodiments, a guide sequence is less than about 75, 50, 45, 40, 35, 30, 25, or 20 nucleotides in length or fewer. Preferably the guide sequence is 10-30 or 15-20 nucleotides long, or 15, 16, 17, 18, 19, or 20 nucleotides in length.

In general, to generate an edit in the target sequence, the gRNA/nuclease or nickase fusion complex binds to a target sequence as determined by the guide RNA, and the nuclease or nickase fusion recognizes a protospacer adjacent motif (PAM) sequence adjacent to the target sequence. The target sequence can be any polynucleotide endogenous or exogenous to the cell, or in vitro. For example, the target sequence can be a polynucleotide residing in the nucleus of the cell. A target sequence can be a sequence encoding a gene product (e.g., a protein) or a non-coding sequence (e.g., a regulatory polynucleotide, an intron, a PAM, a control sequence, or “junk” DNA).

The guide nucleic acid may be and preferably is part of an editing cassette that also encodes the repair template that targets a cellular target sequence. Alternatively, the guide nucleic acid may not be part of the editing cassette and instead may be encoded on the editing vector backbone or the sequences of the gRNA and repair template to be transcribed may be located on separate vectors. For example, a sequence coding for a guide nucleic acid can be assembled or inserted into a vector backbone first, followed by insertion of the repair template in, e.g., an editing cassette. In other cases, the repair template in, e.g., an editing cassette can be inserted or assembled into a vector backbone first, followed by insertion of the sequence coding for the guide nucleic acid. Preferably, the sequence encoding the guide nucleic acid and the repair template are located together in a rationally-designed editing cassette and are simultaneously inserted or assembled via gap repair into a linear plasmid or vector backbone to create an editing vector.

The target sequence is associated with a PAM, which is a short nucleotide sequence recognized by the gRNA/nuclease or nickase fusion complex. The precise preferred PAM sequence and length requirements for different nucleic acid-guided nucleases or nickase fusions vary; however, PAMs typically are 2-7 base-pair sequences adjacent or in proximity to the target sequence and, depending on the nuclease or nickase fusion, can be 5′ or 3′ to the target sequence. Engineering of the PAM-interacting domain of a nucleic acid-guided nuclease or nickase fusion may allow for alteration of PAM specificity, improve target site recognition fidelity, decrease target site recognition fidelity, or increase the versatility of a nucleic acid-guided nuclease or nickase fusion.

In most embodiments, the genome editing of a target sequence both introduces a desired/intended DNA change to a target sequence, e.g., the genomic DNA of a cell, and removes, mutates, or renders inactive a PAM region in the target sequence (e.g., renders the target site immune to further nuclease or nickase fusion binding). Rendering the PAM at the target sequence inactive precludes additional editing of the cell genome at that target sequence, e.g., upon subsequent exposure to a nucleic acid-guided nuclease or nickase fusion complexed with a synthetic guide nucleic acid in later rounds of editing. Thus, in some cells having the desired target sequence edit and an altered PAM can be selected for by using a nucleic acid-guided nuclease or nickase fusion complexed with a synthetic guide nucleic acid complementary to the target sequence, although this is not the case with mammalian cells where double strand breaks are repaired via non-homologous end joining resulting in errors or unmodified sequences. Cells that did not undergo the first editing event will be cut rendering a double-stranded DNA break, and thus will not continue to be viable. The cells containing the desired target sequence edit and PAM alteration will not be cut, as these edited cells no longer contain the necessary PAM site and will continue to grow and propagate.

As for the nuclease or nickase fusion component of the nucleic acid-guided nuclease editing system, a polynucleotide sequence encoding the nucleic acid-guided nuclease or nickase fusion can be codon optimized for expression in particular cell types, such as bacterial, yeast, and, here, mammalian cells. The choice of the nucleic acid-guided nuclease or nickase fusion to be employed depends on many factors, such as what type of edit is to be made in the target sequence and whether an appropriate PAM is located close to the desired target sequence. Nucleases of use in the methods described herein include but are not limited to Cas 9, Cas 12, MAD2, or MAD7, MAD 2007 or other MADzymes and MADzyme systems (see U.S. Pat. No. 910,604,746; 10,655,114; 10,649,754; 10,876,102; 10,833,077; 11,053,485; 10,704,022; 10,745,678; 10,724,021; 10,767,169; 10,870,761; 10,011,849; 10,435,714; 10,626,416; 9,982,279; and 10,337,028; and U.S. Ser. Nos. 16/953,253; 17/374,628; 17/200,074; 17/200,089; 17/200,110; 16/953,233; 17/463,498; 63/134,938; 16/819,896; 17/179,193; and 16/421,783 for sequences and other details related to engineered and naturally-occurring MADzymes).

In specific aspects, the one or more nickases include MAD7 nickase, MAD2001 nickase, MAD2007 nickase, MAD2008 nickase, MAD2009 nickase, MAD2011 nickase, MAD2017 nickase, MAD2019 nickase, MAD297 nickase, MAD298 nickase, MAD299 nickase, or other MAD-series nickases, variants thereof, and/or combinations thereof as described in U.S. Pat. Nos. 10,883,077; 11,053,485; 11,085,030; 11,200,089; 11,193,115; and U.S. Ser. No. 17/463,498. Nickase fusion enzymes typically comprise a CRISPR nucleic acid-guided nuclease engineered to cut one DNA strand in the target DNA rather than making a double-stranded cut, and the nickase portion can be fused to a reverse transcriptase or a nucleotide deaminase as well as other proteins involved in DNA repair, recruitment or nucleotide binding. For more information on nickases and nickase fusion editing, see U.S. Pat. No. 10,689,669 and U.S. Ser. Nos. 16/740,418; 16/740,420 and 16/740,421, all three filed 11 Jan. 2020. Here, a coding sequence for a desired nuclease or nickase fusion is typically on an “engine vector” along with other desired sequences such as a selective marker.

Another component of the nucleic acid-guided nuclease or nickase fusion system is the repair template comprising homology to the target sequence. As described above, the repair template may be on the same vector and even in the same editing cassette as the guide nucleic acid and, if serving as template for a reverse transcriptase, is preferably (but not necessarily) under the control of the same promoter as the gRNA (that is, a single promoter driving the transcription of both the gRNA and the repair template). The template can also be a double stranded or ssDNA donor that is independent from the gRNA sequence. The repair template is designed to serve as a template for homologous recombination or other DNA templated repair with a target sequence in the case of nucleic acid-guided editing or to serve as a template for reverse transcriptase to incorporate the desired edit(s) into the target nucleic acid. A repair template polynucleotide may be of any suitable length, such as about or more than about 20, 25, 50, 75, 100, 150, 200, 500, or 1000 nucleotides in length, and up to 2, 3, 4, 5, 6, 7, 8, 9, 10, 11, 12, 13 and up to 20 kb in length. In certain preferred aspects, the repair template can be provided as an oligonucleotide of between 20-300 nucleotides, more preferably between 50-250 nucleotides. The repair template comprises one or more regions that are complementary to a portion of the target sequence. When optimally aligned, the repair template overlaps with (is complementary to) the target sequence by, e.g., about 20, 25, 30, 35, 40, 50, 60, 70, 80, 90 or more nucleotides. The repair template comprises regions complementary to the target sequence typically flanking the desired edit(s).

As described in relation to the gRNA, the repair template may be provided as part of a rationally-designed editing cassette, which can be inserted into an editing vector (in yeast, preferably a linear vector) where the editing vector may comprise a promoter to drive transcription of the gRNA and, if serving as a template for a reverse transcriptase, the repair template when the editing cassette is inserted into the editing vector. Alternatively, the promoter and gRNA sequence may be separate from repair template and operably linked through complexation or other methods. Moreover, there may be more than one, e.g., two, three, four, or more gRNA/repair template rationally-designed editing cassettes inserted into an editing vector; alternatively, a single rationally-designed editing cassette may comprise two to several gRNA/repair template pairs, where each gRNA is under the control of separate different promoters, separate like promoters, or where all gRNAs/repair template pairs are under the control of a single promoter. In some embodiments the promoter driving transcription of the gRNA and the repair template (or driving more than one gRNA/repair template pair) is optionally an inducible promoter.

In addition to the repair template, an editing cassette may comprise one or more primer sites. The primer sites can be used to amplify the editing cassette by using oligonucleotide primers; for example, if the primer sites flank one or more of the other components of the editing cassette. In addition, the editing cassette may comprise a barcode. A barcode is a unique DNA sequence that corresponds to the repair template sequence such that the barcode can identify the edit made to the corresponding target sequence. The barcode typically comprises four or more nucleotides and is captured to determine what edit was made in the single cell workflow. In some embodiments, the editing cassettes comprise a collection or library of gRNAs and of repair templates representing, e.g., gene-wide or genome-wide libraries of gRNAs and repair templates.

Also, an editing vector or plasmid encoding components of the nucleic acid-guided nuclease or nickase fusion system further encode a nucleic acid-guided nuclease or nickase fusion comprising one or more nuclear localization sequences (NLSs), such as about or more than about 1, 2, 3, 4, 5, 6, 7, 8, 9, 10, or more NLSs, particularly as an element of the nuclease or nickase fusion sequence. In some embodiments, the engineered nuclease or nickase fusion comprises NLSs at or near the amino-terminus, NLSs at or near the carboxy-terminus, or a combination.

### Nucleic Acid-Guided Nickase-Ku or Nickase-Ligase Fusion Protein Genome Editing

The compositions and methods described herein are a “twist” on or alternative to traditional nucleic acid-guided nuclease or nickase fusion editing (i.e., RNA-guided nuclease or CRISPR editing) used to introduce desired edits to a population of live cells; that is, the compositions and methods described herein employ a nucleic acid-guided nuclease-Ku, nuclease-ligase, nickase-Ku or nickase-ligase fusion protein as opposed to a nucleic acid-guided nuclease (i.e., a “CRISPR nuclease”). The nickase-Ku or nickase-ligase fusion employed herein differs from traditional CRISPR editing in that instead of initiating double-strand breaks in the target genome such as with the nuclease-Ku and nuclease-ligase fusions, the nickase fusions initiate a nick in one strand of the target genome. The nickase—having the specificity of a nucleic acid-guided nuclease—engages the gRNA and the target locus, nicks a strand of the target locus and the repair template is incorporated into the nicked strand.

The present disclosure provides compositions of matter, methods and instruments for nucleic acid-guided nuclease-Ku, nuclease-ligase, nickase-Ku or nickase-ligase fusion editing of live cells using a guide RNA with complementarity to, here, the nicked strand at the target locus. With the present compositions and methods, editing efficiency is improved by using fusion proteins (i.e., nickase-Ku or nickase-ligase fusion proteins) that retain the characteristics of nucleic acid-directed nucleases—the binding specificity and ability to cleave one or more DNA strands in a targeted manner—combined with another activity that promotes proper editing. The nucleic acid-guided nuclease-Ku, nuclease-ligase, nickase-Ku or nickase-ligase fusion proteins may be introduced into the cells using a DNA molecule coding for the nucleic acid-guided nuclease-Ku, nuclease-ligase, nickase-Ku or nickase-ligase fusion proteins separately, as part of a ribonucleoprotein (RNP) complex, or encoded on a plasmid or vector, or as mRNA, or in a lentivirus.

The nickase-Ku or nickase-ligase fusions employed herein differ from traditional CRISPR editing in that instead of initiating double-strand breaks in the target genome to effect an edit, the nickase initiates a nick in one strand of the target genome. The fusion of the nickase to a Ku protein or ligase in combination with a sequence appropriate gRNA and sequence-appropriate repair template increases the percentage of edited cells by at least 3-fold and as much as 8-fold.

The present disclosure thus is drawn to increasing the efficiency of nucleic acid-guided nuclease editing, primarily in mammalian cells. Genome editing using nucleic acid-guided nuclease technology requires precise repair of nuclease-induced single or double-strand DNA breaks via homologous recombination or other DNA damage repair mechanisms with a repair template. Double-strand DNA breaks in cells caused by nucleic acid-guided nucleases typically have three main outcomes: 1) cell death if the break is not repaired; 2) non-homologous end joining (NHEJ) which repairs the break without a homologous repair template, and though by-and-large an error-free process, it does have a baseline error rate; and 3) homology-directed recombination (HDR), or donor-templated repair, which uses auxiliary (here, exogenous) homologous DNA—e.g., a repair template sequence—to repair the break.

DNA damage induces several cellular responses that enable the cell to eliminate or deal with the damage or to activate apoptosis (i.e., a programmed cell death process), possibly to eliminate cells with mutations fatal to the cell. DNA damage response reactions include: (a) a transcriptional response, causing changes in the transcription profile that may be beneficial to the cell; (b) removal of DNA damage and rebuilding the DNA duplex; (c) arresting cell cycle progression to allow for repair and to prevent transmission of damaged or incompletely replicated chromosomes; and (d) apoptosis, which excludes heavily damaged or seriously deregulated cells. Mechanisms or DNA repair include direct repair, double-strand break repair, nucleotide excision repair, base excision repair, nucleotide excision repair, and cross-link repair. Nucleic acid-guided nuclease editing, like DNA damage, involves a double-strand break (nuclease) or single-strand nick (nickase).

Mammalian genes that have been implicated in the NHEJ pathway fall into four complementation groups: XRCC4, XRCC5, XRCC6 and XRCC7. XRCC4 protein associates with mammalian DNA ligase IV and may enhance DNA ligase IV activity. XRCC5, XRCC6 and XRCC7 encode components of the DNA-activated protein kinase (DNA-PK), which consists of Ku—which in mammals is a heterodimeric protein—and a catalytic subunit. The mechanism of repair has not been entirely elucidated, however it appears that the Ku heterodimer binds to ends of duplex DNA and, once bound, Ku can translocate internally on the DNA fragment.

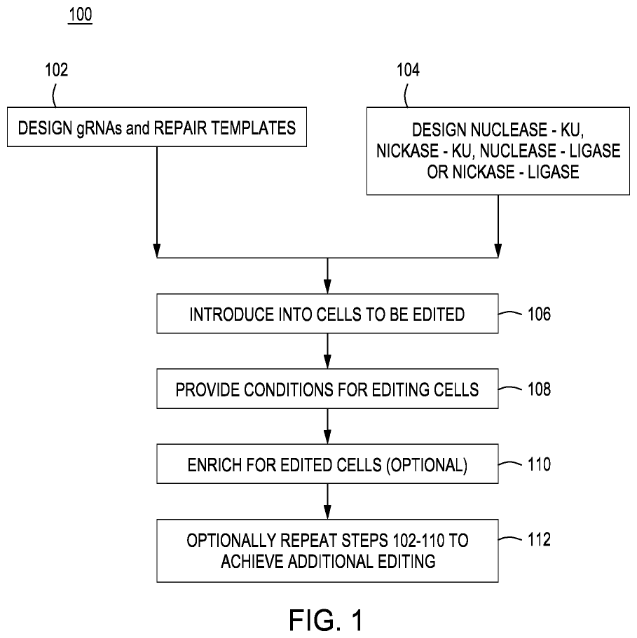

To increase HDR, or donor-templated repair, in nucleic-guided nuclease editing, nucleic acid-guided nuclease-Ku, nuclease-ligase, nickase-Ku or nickase-ligase fusion protein system is employed. FIG. 1 is a simplified block diagram of an exemplary method 100 for editing live cells utilizing one of a nucleic acid-guided nuclease-Ku, nuclease-ligase, nickase-Ku or nickase-ligase fusion protein. The first step 102 in FIG. 1A is to design a library of gRNA/repair template pairs for each desired target region in the genome. The types of edits that can be made using the library of gRNA/repair template pairs are site-directed mutagenesis edits, edits for saturation mutagenesis, promoter swaps and ladders, knock-in and knock-out edits, SNP or short tandem repeat swaps, start/stop codon exchanges, deletions, and any permutation or combination of these types of edits. A library may contain tens, hundreds, thousands, tens of thousands or more different gRNA/repair template pairs; e.g., a library may create tens, hundreds, thousands, tens of thousands or more different types of edits in a single cell population.

In a second step 104, a nucleic acid-guided nuclease-Ku, nuclease-ligase, nickase-Ku or nickase-ligase fusion protein is designed. The nucleic acid nuclease used for the fusion protein can be any nucleic acid-guided nuclease or engineered nickase therefrom including but are not limited to Cas9 (and the H840A nickase), Cas12/CpfI (and any nickase engineered therefrom), MAD2 (and any nickase engineered therefrom), or MAD7 (and the MAD7 nickase, see, e.g., U.S. Pat. No. 10,883,095) or other MADzymes (and any nickase engineered therefrom; see U.S. Pat. No. 10,883,095). For additional nucleases, MADzymes, and nickases see the references above.

In some embodiments of the fusion proteins described herein, the fusions are between a Ku protein or ligase and a nucleic acid-guided nuclease or nickase. Typically, the nuclease or nickase portion comprises the N-terminal portion of the fusion protein and the Ku or ligase portion of the fusion protein comprises the C-terminal portion of the fusion protein, with a linker separating the two portions. Alternatively, the nuclease or nickase portion comprises the C-terminal portion of the fusion protein and the Ku or ligase portion of the fusion protein comprises the N-terminal portion of the fusion protein, with a linker separating the two portions.

Ku is an abundant, highly-conserved DNA binding protein found in both prokaryotes and eukaryotes, which plays an essential role in the maintenance of genome integrity, and is evolutionarily conserved from bacteria to humans. In eukaryotes, Ku is a heterodimer comprised of two subunits, Ku70 (XRCC6) and Ku80 (XRCC5). Ku also is known for a central role in the initial DNA end binding factor in the “classical” non-homologous end joining (C-NHEJ) pathway, which is the main DNA double-strand break (DSB) repair pathway in mammals.

In other embodiments of the nucleic acid-guided nuclease or nickase fusion proteins herein, the nucleic acid-guided nuclease or nickase is fused to a ligase. A nucleic acid ligase is an enzyme that facilitates the joining of DNA strands together by catalyzing the formation of a phosphodiester bond. Ligase plays a role in repairing single-strand breaks in duplex DNA in living organisms, but some forms (such as DNA ligase IV) may specifically repair double-strand breaks. Single-strand breaks are repaired by DNA ligase using the complementary strand of the double helix as a template with DNA ligase creating the final phosphodiester bond to fully repair the DNA. DNA ligase is used in both DNA repair and DNA replication and is used extensively used for recombinant DNA experiments. The mechanism of DNA ligase is to form a covalent phosphodiester bonds between 3′ hydroxyl end of one nucleotide (“acceptor”), with the 5′ phosphate end of another (“donor”).

There are many types of ligases. Bacterial DNA ligases use energy gained by cleaving nicotinamide adenine dinucleotide (NAD) or adenosine triphosphate (ATP) to create the phosphodiester bond. Bacterial DNA ligase D (LigD) is a large protein that participates in nonhomologous end-joining in bacteria. LigD consists of an ATP-dependent ligase domain fused to a polymerase domain (Pol). The Pol activity is depends on manganese, and the ability to perform templated and nontemplated primer extension reactions, and its preference for adding ribonucleotides to blunt DNA ends.

The DNA ligase from bacteriophage T4 (a bacteriophage that infects Escherichia coli bacteria) is commonly used in laboratory research. T4 ligase can ligate either cohesive or blunt ends of DNA, oligonucleotides, as well as RNA and RNA-DNA hybrids, but not single-stranded nucleic acids. T4 ligase can also ligate blunt-ended DNA with much greater efficiency than E. coli and other bacterial DNA ligases. T3 DNA ligase also is an ATP-dependent double-strand DNA ligase from bacteriophage T3. Cohesive ends, blunt ends, and nick sealing are efficiently catalyzed by T3 DNA Ligase.

In addition to phage ligases, there are viral ligases. Chlorella virus PBCV-1 DNA ligase (Chlorella virus DNA ligase), is an ATP-dependent DNA ligase. PBCV-1 DNA ligase efficiently catalyzes the ligation of adjacent, single-stranded DNA ends splinted by a complementary RNA strand. This unusual activity can be used to characterize miRNAs, RNAs and SNPs; that is, sensitive (sub-nanomolar range) detection of a specific RNA by ligation. In addition, PBCV-1 DNA ligase may be used for enrichment of RNA for next-generation sequencing, tolerating all base pair combinations at the ligation junction. Five mammalian DNA ligase activities, I-V, have been purified from mammalian cell extracts and three mammalian LIG genes, where LIG1, LIG3, and LIG4, have been cloned.

The present nucleic acid-guided nuclease-Ku, nuclease-ligase, nickase-Ku or nickase-ligase fusion proteins preferably comprise a linker between the N-terminal and C-terminal proteins. Linkers can be important in the construction of stable, bioactive fusion proteins. The separation distance between functional units can impact epitope access and the ability to bind to a substrate; thus, the availability of a variety of linkers with different lengths and degrees of rigidity are used and are valuable for protein design. Linkers are generally classified into three categories according to their structures: flexible linkers (e.g., (G)ₙ or (GGGGS)ₙ), rigid linkers (e.g., (EAAAK)ₙ), and in vivo cleavable linkers (e.g., disulfide or protease sensitive sequences). In addition to the basic role in linking the functional domains together (as in flexible and rigid linkers) or releasing the free functional domain in vivo (as in in vivo cleavable linkers), linkers offer other advantages for the production of fusion proteins, such as improving biological activity or, as used herein, to combine enzymatic and/or functional (e.g., binding) properties.

Identifying optimal fusion constructs may be accomplished by creating for each nuclease fusion or nickase fusion candidate pair fusions that vary both the length of a linker in between the nuclease or nickase and the Ku or ligase domains and varying the N-terminal and C-terminal positions of the nuclease or nickase and the Ku protein or ligase.

Following design of the gRNA and repair templates 102 and the nuclease fusion or nickase fusion 104 (all of which collectively are “editing components”), the editing components are introduced into the cells to be edited 106. The editing components (including sequences coding for the editing components) may be inserted (via, e.g., Gibson Assembly) into a vector backbone comprising, e.g., a promoter to drive transcription and expression of a nuclease fusion or nickase fusion coding sequence and/or promoter to drive transcription of the gRNA and repair template, as well as one or more origins of replication, and a selective marker such as a gene coding for antibiotic resistance or a cell surface marker. Alternatively, the editing components themselves may be transfected into the cells or the gRNA and nuclease- or nickase-fusion may be transfected into cells as a ribonucleoprotein complex.

The terms transformation and transfection are used interchangeably herein and are intended to include a variety of art-recognized techniques for introducing an exogenous nucleic acid sequence (e.g., engine and/or editing vectors) into a target cell, and the terms “transformation” and “transfection” as used herein include all transformation and transfection techniques. Such methods include, but are not limited to, electroporation, transduction, lipofection, optoporation, injection, microprecipitation, microinjection, liposomes, particle bombardment, sonoporation, laser-induced poration, bead transfection, calcium phosphate or calcium chloride co-precipitation, or DEAE-dextran-mediated transfection. Cells can also be prepared for vector uptake using, e.g., a sucrose, sorbitol or glycerol wash. Additionally, hybrid techniques that exploit the capabilities of mechanical and chemical transfection methods can be used, e.g., magnetofection, a transfection methodology that combines chemical transfection with mechanical methods. In another example, cationic lipids may be deployed in combination with gene guns or electroporators. Suitable materials and methods for transforming or transfecting target cells can be found, e.g., in Green and Sambrook, Molecular Cloning: A Laboratory Manual, 4th Ed., Cold Spring Harbor Laboratory Press, Cold Spring Harbor, N.Y. (2014).

Once the editing components are introduced into the cells 106, the cells are allowed to recover from transformation or transfection, and, optionally, selection may be performed if transformation involves a plasmid or vector comprising a selectable marker. Once recovered and after an optional selection step, conditions are provided for editing the transformed cells 108. The term “providing conditions” means, e.g., to provide chemical or biological agents or temperature or other physical conditions to induce inducible promoters driving transcription of the editing components. If inducible promoters are not used, “providing conditions” typically involves maintaining an optimal temperature and nutrient medium for the transformed cells.

Once enough time has elapsed for the cells to be edited, the cells optionally or preferably are enriched for cells that have been appropriately edited 110. The terms “enrich” or “enrichment” refer to identifying cells that have received the editing components or in which a proper edit results in a specific phenotype or both. Enrichment can be performed directly or using surrogates, e.g., cell surface handles co-introduced with one or more components of the editing components. Selectable markers, such as genes conferring antibiotic resistance may be used; alternatively, optical selectable markers such as fluorescent proteins (e.g., green, red and/or blue fluorescent proteins) may be used.

At this point in method 100, the cells can be characterized phenotypically or genotypically or optionally steps 102-110 may be repeated to make additional edits 112.

### Compositions Perform Nucleic Acid-Guided Nuclease or Nickase Fusion Editing in Cells in an Automated Device

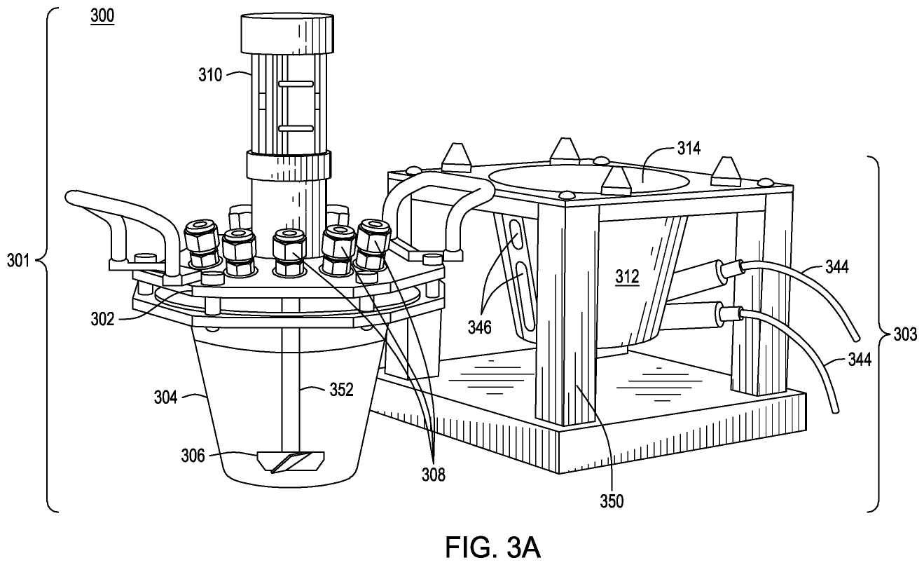

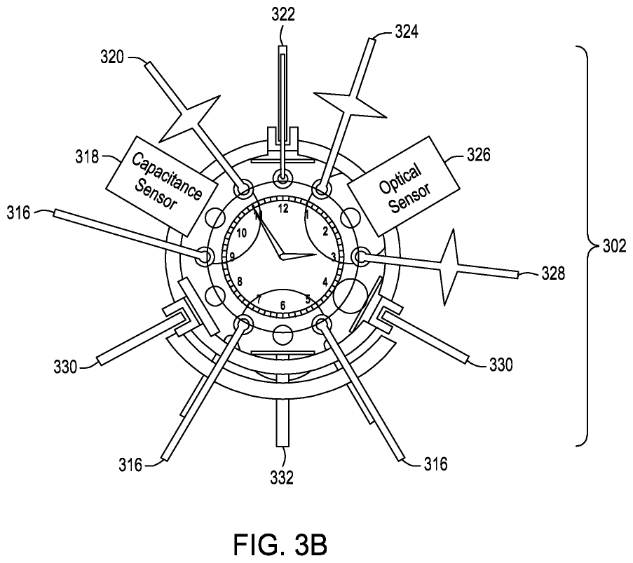

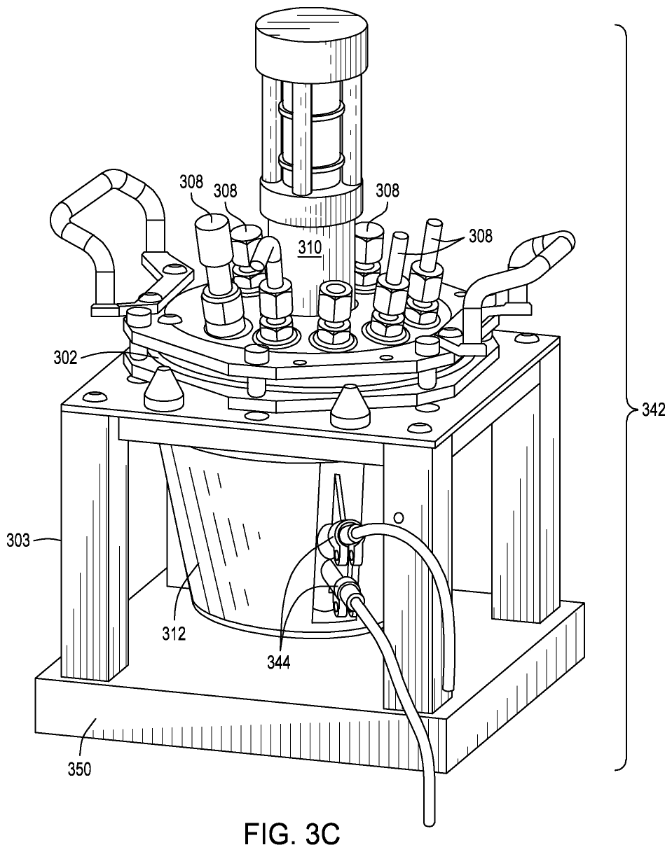

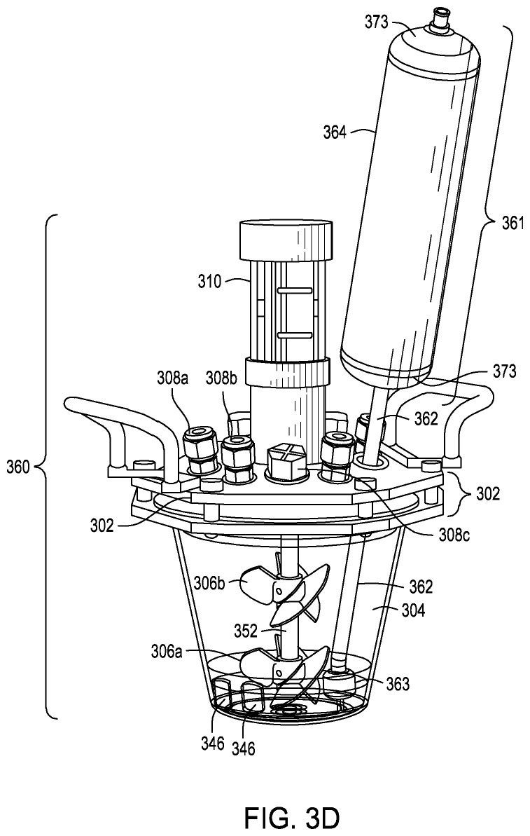

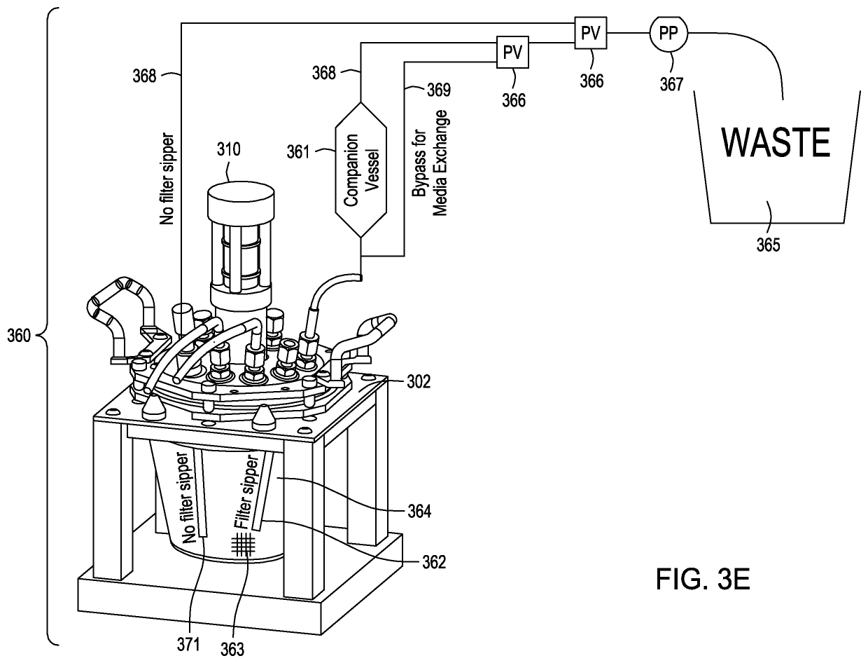

In the editing methods described herein, cells, such as, in one embodiment, stem cells to be edited are grown for several passages, e.g., off instrument, to assure cell health. The cells may be grown in 2D culture, in 3D culture (if the cells are viable when grown in or adapted to 3D culture) or on microcarriers. This initial cell growth typically takes place off the automated instrument (the instrument is described infra in relation to FIGS. 3A-3E). If necessary, the cells are dissociated and added to medium in the bioreactor comprising cell growth medium such as MEM, DMEM, RPMI, or, for stem cells, mTeSRTMPlus serum-free, feeder-free cell culture medium (STEMCELL Technologies Canada INC., Vancouver, BC) and cell growth microcarriers. If the cells are grown initially on microcarriers, the microcarriers are transferred to the bioreactor comprising cell growth medium such as mTeSRTMPlus serum-free, feeder-free cell culture medium and additional microcarriers. Approximately 1e7 or 1e8 cells are transferred to the cell growth module on the automated instrument for growth.

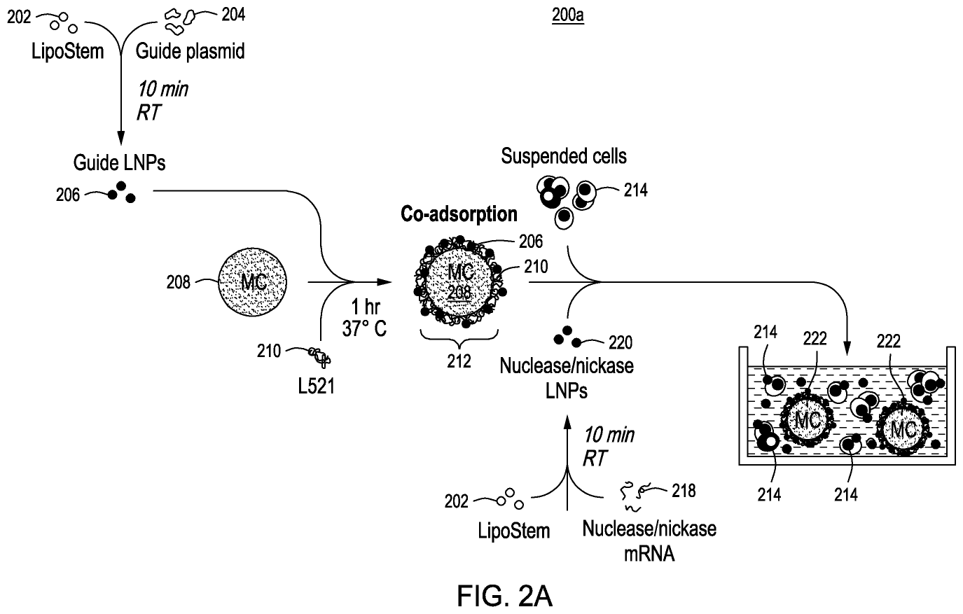

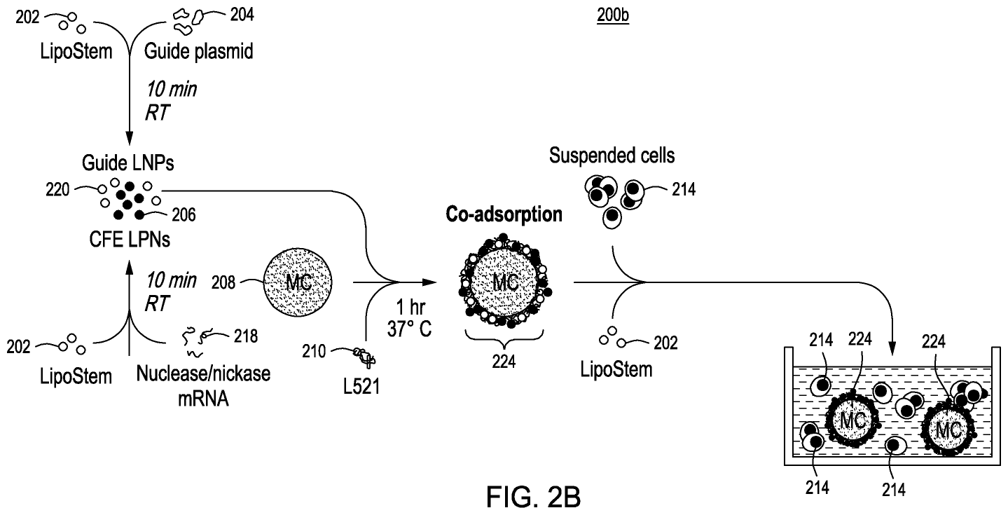

In parallel with the off-instrument cell growth, reagent bundle microcarriers (RBMCs) are manufactured, also off-instrument. The present description provides depictions of two exemplary methods for manufacturing RBMCs (see FIGS. 2A and 2B) that may be used to edit the cells in the modules and automated instruments described herein.

The cells are grown in 3D culture on microcarriers in the bioreactor for, e.g., three to four days or until a desired number of cells, e.g., 1e8, cells are present. These processes may take place in the bioreactor and cell corral (described infra). During this growth cycle, the cells are monitored for cell number, pH, and optionally other parameters. As described above, cell growth monitoring can be performed by imaging, for example, by allowing the microcarriers to settle and imaging the bottom of the bioreactor. Alternatively, an aliquot of the culture may be removed and run through a separate flow cell, e.g., in a separate module, for imaging. For example, the cell corral, in addition to being integrated with the bioreactor vessel, may be integrated with a flow cell or other device for cell counting where an aliquot of the cell culture in the cell corral may be removed and counted in the flow cell. In another alternative, the cells may express a fluorescent protein and fluorescence in the cell culture is measured or fluorescent dye may be used to stain cells, particularly live cells. This microcarrier-based workflow can be performed in the bioreactor and cell corral with most if not all steps performed in the same device; thus, several bioreactors and cell corrals may be deployed in parallel for two to many samples simultaneously. In yet another alternative, permittivity or capacitance is used to monitor cell coverage on the microcarriers. In yet another embodiment, an aliquot of cells may be removed from the bioreactor or cell corral and transported out of the instrument and manually counted on a commercial cell counter (i.e., Thermofisher Countess, Waltham, Mass., USA). Cell aliquots from the stem cell culture to be used to monitor pluripotency may be removed via “liquid out” ports in the bioreactor

The microcarriers used for initial cell growth can be nonporous (where pore sizes are typically <20 nm in size), microporous (with pores between >20 nm to <1 μm in size), or macroporous (with pores between >1 μm in size, e.g. 20 μm). In microcarrier culture, cells grow as monolayers on the surface of nonporous or microporous microcarriers, which are typically spherical in morphology; alternatively, the cells grow on the surface and as multilayers in the pores of macroporous microcarriers. The microcarriers preferably have a density slightly greater than that of the culture medium to facilitate easy separation of cells and medium for, e.g., medium exchange and imaging and passaging; yet the density of the microcarriers is also sufficiently low to allow complete suspension of the microcarriers at a minimum stirring or bubbling rate. Maintaining a low stirring or bubbling rate is preferred so as to avoid hydrodynamic damage to the cells.

The microcarriers used for cell growth depend on cell type and desired cell numbers, and typically include a coating of a natural or synthetic extracellular matrix or cell adhesion promoters (e.g., antibodies to cell surface proteins or poly-L-lysine) to promote cell growth and adherence. Microcarriers for cell culture are widely commercially available from, e.g., Millipore Sigma (St. Louis, Mo., USA); Thermo Fisher (Waltham, Mass., USA); Pall Corp. (Port Washington, N.Y., USA); GE Life Sciences (Marlborough, Mass., USA); and Corning Life Sciences (Tewkesbury, Mass., USA). As for the extracellular matrix, natural matrices include collagen, fibrin and vitronectin (available, e.g., from ESBio, Alameda, Calif., USA), and synthetic matrices include Matrigel® (Corning Life Sciences, Tewkesbury, Mass., USA), Geltrex™ (Thermo Fisher Scientific, Waltham, Mass., USA), Cultrex® (Trevigen, Gaithersburg, Md., USA), biomemetic hydrogels available from Cellendes (Tubingen, Germany); and tissue-specific extracellular matrices available from Xylyx (Brooklyn, N.Y., USA); further, denovoMatrix (Dresden, Germany) offers screen MATRIX™, a tool that facilitates rapid testing of a large variety of cell microenvironments (e.g., extracellular matrices) for optimizing growth of the cells of interest.

Following cell growth, passaging is performed by, e.g., stopping the impeller rotation or bubbling action in the bioreactor and allowing the microcarriers to settle. In one method, the cells are removed from the microcarriers using enzymes such as collagenase, trypsin or pronase, or by non-enzymatic methods including EDTA or other chelating chemicals, and once removed from the carriers, medium is added to dilute the enzyme to inhibit enzymatic action. The dissociation procedures relating to the cell corral are described in detail infra. Once medium is added, then the cells are separated from the microcarriers by allowing the microcarriers to settle and aspirating the cells via a filtered sipper into the cell corral. The cells then may be optionally dissociated from one another via a filter, sieve or by bubbling or other agitation in the cell corral and aliquots removed, e.g., for pluripotency determination. Next, microcarriers comprising the manufactured reagent bundles (RBMCs) and the dissociated cells are combined in an appropriate medium in the growth vessel. Alternatively, instead of removing cells from the cell growth microcarriers and re-seeding on RBMCs, the cells may be transferred from the cell growth microcarriers to RBMCs via microcarrier bridge passaging either in the growth vessel in a reduced volume or in the cell corral. Bridge passaging involves allowing a new microcarrier (e.g. an RBMC) to come into physical contact with a cell-laden microcarrier, such that cells on the latter microcarrier can migrate to the RBMC.

RBMCs are not prepared on-instrument but are pre-manufactured. The microcarriers used for reagent bundles may be microporous microcarriers, which, due to the plethora of micropores, can carry a larger reagent payload per carrier diameter than nonporous or macroporous microcarriers. Preferred RBMCs are microporous, to provide increased surface area for reagent delivery, and functionalized on the surface so as to be able to bind reagents. Preferred microcarriers for RBMCs include Pierce™ Streptavidin UltraLink™ Resin, a cross-linked polyacrylamide carrier functionalized with streptavidin comprising a pore size of 50 to 100 nm; Pierce™ NeutrAvidin™ Plus UltraLink™ Resin, cross-linked polyacrylamide carrier functionalized with avidin comprising a pore size of 50 to 100 nm; and UltraLink™ Hydrazide Resin, a cross-linked polyacrylamide carrier functionalized with hydrazine comprising a pore size of 50 to 100 nm, all available from Thermo Fisher (Waltham, Mass., USA); cross-linked agarose resins with alkyne, azide, photo-cleavable azide and disulfide surface functional groups available from Click Chemistry Tools (Scottsdale, Ariz., USA); Sepharose™ Resin, cross-linked agarose with amine, carboxyl, carbodiimide, N-hydroxysuccinimide (NHS), and epoxy surface functional groups available from GE Health (Chicago, Ill., USA).

The microcarriers are loaded with amplified editing cassettes or amplified editing plasmids, engine plasmids, nuclease or nuclease fusion proteins, mRNAs or ribonucleoproetins (RNPs) depending on, e.g., the functionalized group, via, e.g., via chemical or photo linkage or depending on a surface coating on the microcarrier, if present. RBMCs are prepared by 1) partitioning and amplifying a single copy of an editing cassette to produce clonal copies in an RBMC, or by 2) pooling and amplifying editing cassettes, followed by dividing the editing cassettes into sub-pools and “pulling down” the amplified editing cassettes with microcarriers comprising nucleic acids specific to and complementary to unique sequences on the editing cassettes. The step of sub-pooling acts to “de-multiplex” the editing cassette pool, thereby increasing the efficiency and specificity of the “pull down” process. De-multiplexing thus allows for amplification and error correction of the editing cassettes to be performed in bulk followed by efficient loading of clonal copies of the editing cassettes onto a microcarrier.

An exemplary option for growing, passaging, transfecting and editing iPSCs (induced pluripotent stem cells), where there is sequential delivery of clonal high copy number (HCN) RBMCs, i.e., lipid nanoparticle-coated microcarriers, where each microcarrier is coated with many copies of delivery vehicles (e.g., RNA, DNA, plasmid, or ribonucleoprotein) carrying a single clonal editing cassette-followed by bulk enzyme delivery. Note that the bioreactors and cell corrals described infra may be used for all processes. First, cells are seeded on the RBMCs to deliver clonal copies of nucleic acids to the cells. Again, the RBMCs are typically fabricated or manufactured off-instrument. The cells are allowed to grow and after 24-48 hours, medium is exchanged for medium containing antibiotics to select for cells that have been transfected. The cells are passaged, re-seeded and grown again, and then passaged and re-seeded, this time onto microcarriers comprising lipofectamine with the enzyme provided as a coding sequence under the control of a promoter, or as a protein on the surface of a microcarrier. As an alternative, the enzyme may be provided in bulk in solution. The enzyme is taken up by the cells on the microcarriers, and the cells are incubated and allowed to grow. Medium is exchanged as needed and the cells are detached from the microcarriers for subsequent growth and analysis.

An alternative exemplary option comprises the steps of growing, passaging, transfecting and editing iPSCs. In this embodiment, there is simultaneous delivery of clonal high copy number (HCN) RBMCs (i.e., reagent bundle lipid nanoparticle-coated microcarriers) where each microcarrier is coated with many copies of delivery vehicles (e.g., RNA, DNA, plasmid, or ribonucleoprotein) carrying a single clonal editing cassette and enzyme (e.g., as a coding sequence under the control of a promoter therefor, as a ribonucleoprotein complex, or as a protein). Again, the RBMCs are typically fabricated or manufactured off-instrument. Note that the integrated instrument described infra may be used for all processes. As with the workflow described above, first cells are seeded on microcarriers to grow. The cells are then passaged, detached, re-seeded, grown and detached again to increase cell number, with medium exchanged every 24-72 hours as needed. Following detachment, the cells are seeded on RBMCs for clonal delivery of the editing cassette and enzyme in a co-transfection reaction. Following transfection, the cells grown for 24-48 hours after which medium is exchanged for medium containing antibiotics for selection. The cells are selected and passaged, re-seeded and grown again. Medium is exchanged as needed and the cells are detached from the microcarriers for subsequent growth and analysis.

FIGS. 2A and 2B depict alternative methods for populating microcarriers with a lipofectamine/nucleic acid payload and cells. In the method 200a shown in FIG. 2A at top left, lipofectamine 202 and guide plasmid (comprising the gRNA and repair template pairs) or guide plasmid and donor DNA which are complexed through other means, payloads 204 are combined and guide/repair template LNPs (lipofectamine nucleic acid payloads) 206 are formed in solution. In parallel, microcarriers 208 (“MCs”) are combined with a coating such as laminin 521 210 to foster adsorption and cell attachment. The laminin 521-coated microcarriers are then combined with the guide/repair template LNPs 206 to form partially-loaded microcarriers 212. The processes of forming RBMCs (i.e., the partially-loaded microcarriers 212 comprising the guide/repair template LNPs 206) to this point are typically performed off-instrument. In parallel and typically off-instrument, nuclease- or nickase-Ku or nuclease- or nickase-ligase LNPs (or nuclease or nickase LNPs) 220 are formed by combining lipofectamine 202 and nuclease-Ku, nuclease-ligase, nickase-Ku or nickase-ligase mRNA 218. The nuclease or nickase LNPs 220 are combined with the partially-loaded microcarriers 212 and adsorb onto the partially-loaded microcarriers 212 to form fully-loaded RBMCs 222 comprising both the guide/repair template LNPs 206 and the nuclease or nickase LNPs 220. At this point, the stem cells 214 have been grown and passaged in the bioreactor and cell corral several to many times. The cells 214 populate the fully-loaded RBMCs 222, where the cells 214 then take up (i.e., are transfected by) the guide/repair template LNPs 206 and the nuclease or nickase LNPs 220, a process that may take several hours up to several days. At the end of the transfection process, transfected stem cells reside on the surface of the fully-loaded microcarriers 222.

As an alternative to the method 200a shown in FIG. 2A, FIG. 2B depicts method 200b which features simultaneous adsorption of the guide/repair template LNPs and the nuclease/nickase LNPs. Again, lipofectamine 202 and guide plasmid payloads 204 are combined where guide/repair template LNPs (lipofectamine nucleic acid payloads) 206 are formed in solution. In parallel, nuclease or nickase LNPs 220 are formed by combining lipofectamine 202 and nuclease or nickase mRNA 218. Also in parallel, microcarriers 208 are combined with a coating such as laminin 521 210 to foster adsorption and cell attachment. The laminin 521-coated microcarriers are simultaneously combined with both the guide/repair template LNPs 206 and the nuclease or nickase LNPs 220 to form fully-loaded microcarriers 224 where both the guide/repair template LNPs 206 and the nuclease or nickase LNPs 220 co-adsorb onto the surface of the laminin-coated microcarriers. The processes of forming RBMCs (i.e., the fully-loaded microcarriers 224 comprising both the guide/repair template LNPs 206 and the nuclease or nickase LNPs 220) to this point are typically performed off-instrument.

At this point, the fully-loaded microcarriers 224 comprising the guide/repair template LNPs 206 and the nuclease or nickase LNPs 220 are added to medium in the bioreactor comprising the stem cells 214 to be transfected, optionally with additional lipofect reagent 202. The stem cells 214 have been grown and passaged in the bioreactor and cell corral one to many times. The cells 214 populate the fully-loaded RBMCs 224, where the cells 214 then take up (i.e., are transfected by) the guide/repair template LNPs 206 and the nuclease or nickase LNPs 220, a process that may take several hours up to several days. At the end of the transfection process, transfected stem cells reside on the surface of the fully-loaded microcarriers 224. In these exemplary methods, nuclease or nickase fusion mRNAs are used to form the nuclease/nickase LNPs; however, the nuclease or nickase enzymes may be loaded on to form LNPs, or gRNAs and nuclease or nickase enzymes may be loaded in the form of RNPs on the LNPs.

A bioreactor may be used to grow cells—in particular mammalian cells—off-instrument or to allow for cell growth and recovery on-instrument; e.g., as one module of a fully-automated closed instrument. Further, the bioreactor supports cell selection/enrichment, via expressed antibiotic markers in the growth process or via expressed antibodies coupled to magnetic beads and a magnet associated with the bioreactor. There are many bioreactors known in the art, including those described in, e.g., WO2019/046766; U.S. Pat. No. 10,699,519; 10,633,625; 10,577,576; 10,294,447; 10,240,117; 10,179,898; 10,370,629; and 9,175,259; and those available from Lonza Group Ltd. (Basel, Switzerland); Miltenyi Biotec (Bergisch Gladbach, Germany), Terumo BCT (Lakewood, Colo., USA) and Sartorius GmbH (Gottingen, Germany).

FIG. 3A shows one embodiment of a bioreactor assembly 300 suitable for cell growth, transfection, and editing as one component of an automated cell processing instrument. Unlike most bioreactors that are used to support fermentation or other processes with an eye to harvesting the products produced by organisms grown in the bioreactor, the present bioreactor (and the processes performed therein) is configured to grow cells, monitor cell growth (via, e.g., optical means or capacitance), passage cells, select cells, transfect cells, and support the growth and harvesting of edited cells. Bioreactor assembly 300 comprises cell growth vessel 301 comprising a main body 304 with a lid assembly 302 comprising ports 308, including a motor integration port 310 configured to accommodate a motor to drive impeller 306 via impeller shaft 352. The tapered shape of main body 304 of the growth vessel 301 along with, in some embodiments, dual impellers allows for working with a larger dynamic range of volumes, such as, e.g., up to 500 ml and as low as 100 ml for rapid sedimentation of the microcarriers.

Bioreactor assembly 300 further comprises bioreactor stand assembly 303 comprising a main body 312 and growth vessel holder 314 comprising a heat jacket or other heating means (not shown) into which the main body 304 of growth vessel 301 is disposed in operation. The main body 304 of growth vessel 301 is biocompatible and preferably transparent—in some embodiments, in the UV and IR range as well as the visible spectrum—so that the growing cells can be visualized by, e.g., cameras or sensors integrated into lid assembly 302 or through viewing apertures or slots 346 in the main body 312 of bioreactor stand assembly 303. Camera mounts are shown at 344.

Bioreactor assembly 300 supports growth of cells from a 500,000 cell input to a 10 billion cell output, or from a 1 million cell input to a 25 billion cell output, or from a 5 million cell input to a 50 billion cell output or combinations of these ranges depending on, e.g., the size of main body 304 of growth vessel 301, the medium used to grow the cells, the type and size and number of microcarriers used for growth (if microcarriers are used), and whether the cells are adherent or non-adherent. The bioreactor that comprises assembly 300 supports growth of both adherent and non-adherent cells, wherein adherent cells are typically grown of microcarriers as described in detail in U.S. Ser. No. 17/237,747, filed 24 Apr. 2021. Alternatively, another option for growing mammalian cells in the bioreactor described herein is growing single cells in suspension using a specialized medium such as that developed by ACCELLTA™ (Haifa, Israel). Cells grown in this medium must be adapted to this process over many cell passages; however, once adapted the cells can be grown to a density of >40 million cells/ml and expanded 50-100× in approximately a week, depending on cell type.

Main body 304 of growth vessel 301 preferably is manufactured by injection molding, as is, in some embodiments, impeller 306 and the impeller shaft 352. Impeller 306 also may be fabricated from stainless steel, metal, plastics or the polymers listed infra. Injection molding allows for flexibility in size and configuration and also allows for, e.g., volume markings to be added to the main body 304 of growth vessel 301. Additionally, material from which the main body 304 of growth vessel 301 is fabricated should be able to be cooled to about 4° C. or lower and heated to about 55° C. or higher to accommodate cell growth. Further, the material that is used to fabricate the vial preferably is able to withstand temperatures up to 55° C. without deformation. Suitable materials for main body 304 of growth vessel 301 include cyclic olefin copolymer (COC), glass, polyvinyl chloride, polyethylene, polyetheretherketone (PEEK), polypropylene, polycarbonate, poly(methyl methacrylate (PMMA)), polysulfone, poly(dimethylsiloxane), cyclo-olefin polymer (COP), and co-polymers of these and other polymers. Preferred materials include polypropylene, polycarbonate, or polystyrene. The material used for fabrication may depend on the cell type to be grown, transfected and edited, and be conducive to growth of both adherent and non-adherent cells and workflows involving microcarrier-based transfection. The main body 304 of growth vessel 301 may be reusable or, alternatively, may be manufactured and configured for a single use. In one embodiment, main body 304 of growth vessel 301 may support cell culture volumes of 25 ml to 500 ml, but may be scaled up to support cell culture volumes of up to 3 L.

The bioreactor stand assembly comprises a stand or frame 350 and a main body 312 that holds the growth vessel 301 during operation. The stand/frame 350 and main body 312 are fabricated from stainless steel, other metals, or polymer/plastics. The bioreactor stand assembly main body further comprises a heat jacket (not seen in FIG. 3A) to maintain the growth vessel main body 304—and thus the cell culture—at a desired temperature. Additionally, the stand assembly can host a set of sensors and cameras (camera mounts are shown at 344) to monitor cell culture.

FIG. 3B depicts a top-down view of one embodiment of vessel lid assembly 302. Growth vessel lid assembly 302 is configured to be air-tight, providing a sealed, sterile environment for cell growth, transfection and editing as well as to provide biosafety in a closed system. Vessel lid assembly 302 and the main body of growth vessel can be reversibly sealed via fasteners such as screws, or permanently sealed using biocompatible glues or ultrasonic welding. Vessel lid assembly 302 in some embodiments is fabricated from stainless steel such as S316L stainless steel but may also be fabricated from metals, other polymers (such as those listed supra) or plastics. As seen in this FIG. 3B—as well as in FIG. 3A—vessel lid assembly 302 comprises a number of different ports to accommodate liquid addition and removal; gas addition and removal; for insertion of sensors to monitor culture parameters (described in more detail infra); to accommodate one or more cameras or other optical sensors; to provide access to the main body 304 of growth vessel 301 by, e.g., a liquid handling device; and to accommodate a motor for motor integration to drive one or more impellers 306. Exemplary ports depicted in FIG. 3B include three liquid-in ports 316 (at 5 o'clock, 7 o'clock and 9 o'clock); two self-sealing ports 330 (at 4 o'clock and at 8 o'clock) to provide access to the main body 304 of growth vessel 301; one liquid-out port 322 (at 12 o'clock); a capacitance sensor 318 (at 10 o'clock); one “gas in” port 324 (at 1 o'clock); one “gas out” port 320 (at 11 o'clock); an optical sensor 326 (at 2 o'clock); a rupture disc 328 at 3 o'clock; and (a temperature probe 332 (at 6 o'clock). [Note the clock face is for reference only.]

The ports shown in vessel lid assembly 302 in this FIG. 3B are exemplary only and it should be apparent to one of ordinary skill in the art given the present disclosure that, e.g., a single liquid-in port 316 could be used to accommodate addition of all liquids to the cell culture rather than having a liquid-in port for each different liquid added to the cell culture. Further, any liquid-in port may serve as both a liquid-in port and a liquid-out port. Similarly, there may be more than one gas-in port 324, such as one for each gas, e.g., O₂, CO₂ that may be added. In addition, although a temperature probe 332 is shown, a temperature probe alternatively may be located on the outside of vessel holder 314 of bioreactor stand assembly 303 separate from or integrated into heater jacket (not seen in this FIG. 3B). A self-sealing port 330, if present, allows access to the main body 304 of growth vessel 301 for, e.g., a pipette, syringe, or other liquid delivery system via a gantry (not shown). As shown in FIG. 3A, additionally there may be a motor integration port 310 to drive the impeller(s), although other configurations of growth vessel 301 may alternatively integrate the motor drive at the bottom of the main body 304 of growth vessel 301. Growth vessel lid assembly 302 may also comprise a camera port for viewing and monitoring the cells.

Additional sensors include those that detect dissolved O₂ concentration, dissolved CO₂ concentration, culture pH, lactate concentration, glucose concentration, biomass, and optical density. The sensors may use optical (e.g., fluorescence detection), electrochemical, or capacitance sensing and either be reusable or configured and fabricated for single-use. Sensors appropriate for use in the bioreactor are available from Omega Engineering (Norwalk, Conn., USA); PreSens Precision Sensing (Regensburg, Germany); C-CIT Sensors AG (Waedenswil, Switzerland), and ABER Instruments Ltd. (Alexandria, Va., USA). In one embodiment, optical density is measured using a reflective optical density sensor to facilitate sterilization, improve dynamic range and simplify mechanical assembly.

The rupture disc, if present, provides safety in a pressurized environment, and is programmed to rupture if a threshold pressure is exceeded in growth vessel. If the cell culture in the growth vessel is a culture of adherent cells, microcarriers may be used as described in U.S. Ser. No. 17/237,747, filed 24 Apr. 2021. In such an instance, the liquid-out port may comprise a filter such as a stainless steel or plastic (e.g., polyvinylidene difluoride (PVDF), nylon, polypropylene, polybutylene, acetal, polyethylene, or polyamide) filter or frit to prevent microcarriers from being drawn out of the culture during, e.g., medium exchange, but to allow dead cells to be withdrawn from the vessel. Additionally, a liquid port may comprise a filter sipper to allow cells that have been dissociated from microcarriers to be drawn into the cell corral while leaving spent microcarriers in main body 304 of growth vessel 301. The microcarriers used for initial cell growth can be nanoporous (where pore sizes are typically <20 nm in size), microporous (with pores between >20 nm to <1 μm in size), or macroporous (with pores between >1 μm in size, e.g. 20 μm) and the microcarriers are typically 50-200 μm in diameter; thus the pore size of the filter or frit in the liquid-out port will differ depending on microcarrier size.

The microcarriers used for cell growth depend on cell type and desired cell numbers, and typically include a coating of a natural or synthetic extracellular matrix or cell adhesion promoters (e.g., antibodies to cell surface proteins or poly-L-lysine) to promote cell growth and adherence. Microcarriers for cell culture are widely commercially available from, e.g., Millipore Sigma, (St. Louis, Mo., USA); ThermoFisher Scientific (Waltham, Mass., USA); Pall Corp. (Port Washington, N.Y., USA); GE Life Sciences (Marlborough, Mass., USA); and Corning Life Sciences (Tewkesbury, Mass., USA). As for the extracellular matrix, natural matrices include collagen, fibrin and vitronectin (available, e.g., from ESBio, Alameda, Calif., USA), and synthetic matrices include MATRIGEL® (Corning Life Sciences, Tewkesbury, Mass., USA), GELTREX™ (ThermoFisher Scientific, Waltham, Mass., USA), CULTREX® (Trevigen, Gaithersburg, Md., USA), biomemetic hydrogels available from Cellendes (Tubingen, Germany); and tissue-specific extracellular matrices available from Xylyx (Brooklyn, N.Y., USA); further, denovoMatrix (Dresden, Germany) offers screenMATRIX™, a tool that facilitates rapid testing of a large variety of cell microenvironments (e.g., extracellular matrices) for optimizing growth of the cells of interest.

FIG. 3C is a side perspective view of the assembled bioreactor 342 without sensors mounted in ports 308. Seen are vessel lid assembly 302, bioreactor stand assembly 303, bioreactor stand main body 312 into which the main body of growth vessel 301 (not seen in this FIG. 3C) is inserted. Also present are two camera mounts 344, a motor integration port 310, and stand or frame 350.

FIG. 3D shows the embodiment of a bioreactor/cell corral assembly 360, comprising the bioreactor assembly 300 for cell growth, transfection, and editing described in FIG. 3A and further comprising a cell corral 361. Bioreactor assembly 300 comprises a growth vessel 301 (not labeled in this FIG. D) comprising tapered a main body 304 with a lid assembly 302 comprising ports 308 (here, 308a, 308b, 308c), including a motor integration port 310 driving impellers 306a and 306b via impeller shaft 352, as well as two viewing ports 346. Cell corral 361 comprises a main body 364, and end caps 373, where the end cap proximal the bioreactor assembly 300 is coupled to a filter sipper 362 comprising a filter portion 363 disposed within the main body 304 of the bioreactor assembly 300. The filter sipper is disposed within the main body 304 of the bioreactor assembly 300 but does not reach to the bottom surface of the bioreactor assembly 300 to leave a “dead volume” for spent microcarriers to settle while cells are removed from the growth vessel 301 into the cell corral 361. The cell corral may or may not comprise a temperature or CO₂ probe, and may or not be enclosed within an insulated jacket.

The cell corral 361, like the main body 304 of growth vessel 301 is fabricated from any biocompatible material such as polycarbonate, cyclic olefin copolymer (COC), glass, polyvinyl chloride, polyethylene, polyetheretherketone (PEEK), polypropylene, poly(methyl methacrylate (PMMA)), polysulfone, poly(dimethylsiloxane), cyclo-olefin polymer (COP), and co-polymers of these and other polymers. Likewise, the end caps 373 of the cell corral are fabricated from a biocompatible material such as polycarbonate, cyclic olefin copolymer (COC), glass, polyvinyl chloride, polyethylene, polyetheretherketone (PEEK), polypropylene, poly(methyl methacrylate (PMMA)), polysulfone, poly(dimethylsiloxane), cyclo-olefin polymer (COP), and co-polymers of these and other polymers. The cell corral may be coupled to or integrated with one or more devices, such as a flow cell where an aliquot of the cell culture can be counted. Additionally, the cell corral may comprise additional liquid ports for adding medium, other reagents, and/or fresh microcarriers to the cells in the cell corral. The volume of the main body 364 of the cell corral 361 may be from 25 to 3000 mL, or from 250 to 1000 mL, or from 450 to 500 mL.

In operation, the bioreactor/cell corral assembly 360 comprising the bioreactor assembly 300 and cell corral 361 grows, passages, transfects, and supports editing and further growth of mammalian cells (note, the bioreactor stand assembly is not shown in this FIG. 3D). Cells are transferred to the growth vessel 301 comprising medium and microcarriers. The cells are allowed to adhere to the microcarriers. Approximately 2000,000 microcarriers (e.g., laminin-521 coated polystyrene with enhanced attachment surface treatment) are used for the initial culture of approximately 20 million cells to where there are approximately 50 cells per microcarrier. The cells are grown until there are approximately 500 cells per microcarrier. For medium exchange, the microcarriers comprising the cells are allowed to settle and spent medium is aspirated via a sipper filter, wherein the filter has a mesh small enough to exclude the microcarriers. The mesh size of the filter will depend on the size of the microcarriers and cells present but typically is from 50 to 500 μm, or from 70 to 200 μm, or from 80 to 110 μm. For passaging the cells, the microcarriers are allowed to settle and spent medium is removed from the growth vessel 301, and phosphobuffered saline or another wash agent is added to the growth vessel 301 to wash the cells on the microcarriers. Optionally, the microcarriers are allowed to settle once again, and some of the wash agent is removed. At this point, the cells are dissociated from the microcarriers. Dissociation may be accomplished by, e.g., bubbling gas or air through the wash agent in the growth vessel 301, by increasing the impeller speed and/or direction, by enzymatic action (via, e.g., trypsin), or by a combination of these methods. In one embodiment, a chemical agent such as the RelesR™ reagent (STEMCELL Technologies Canada INC., Vancouver, BC, Canada) is added to the microcarriers in the remaining wash agent for a period of time required to dissociate most of the cells from the microcarriers, such as from 1 to 60 minutes, or from 3 to 25 minutes, or from 5 to 10 minutes. Once enough time has passed to dissociate the cells, cell growth medium is added to the growth vessel 301 to stop the enzymatic reaction.

Once again, the now-spent microcarriers are allowed to settle to the bottom of the growth vessel 301 and the cells are aspirated through a filter sipper into the cell corral 361. The growth vessel 301 is configured to allow for a “dead volume” of 2 mL to 200 mL, or 6 mL to 50 mL, or 8 mL to 12 mL below which the filter sipper does not aspirate medium to ensure the settled spent microcarriers are not transported to the filter sipper during fluid exchanges. Once the cells are aspirated from the bioreactor vessel leaving the “dead volume” of medium and spent microcarriers, the spent microcarriers are aspirated through a non-filter sipper into waste. The spent microcarriers (and the bioreactor vessel) are diluted in phosphobuffered saline or other buffer one or more times, wherein the wash agent and spent microcarriers continue to be aspirated via the non-filter sipper leaving a clean bioreactor vessel. After washing, fresh microcarriers or RBMCs and fresh medium are dispensed into the bioreactor vessel and the cells in the cell corral are dispensed back into the bioreactor vessel for another round of passaging or for transfection and editing, respectively.

FIG. 3E depicts a bioreactor and bioreactor/cell corral assembly 360 comprising a growth vessel 301 (not shown in this FIG. 3E), with a main body 364, lid assembly 302 comprising a motor integration port 310, a filter sipper 362 comprising a filter 363 and a non-filter sipper 371, 368. Also seen is a cell corral 361, fluid line from the cell corral through pinch valve 366, and a line 369 for medium exchange also connected to a pinch valve 366. The non-filter sipper 368 also runs through a pinch valve 366 to waste 365. Also seen is a peristaltic pump 367.

It should be apparent to one of ordinary skill in the art given the present disclosure that the process described may be recursive and multiplexed; that is, cells may go through the method described in relation to FIG. 1, then the resulting edited culture may go through another (or several or many) rounds of additional editing (e.g., recursive editing) with different editing components. For example, the cells from round 1 of editing may be diluted and an aliquot of the edited cells edited by editing components A may be combined with editing components B, an aliquot of the edited cells edited by editing components A may be combined with editing components C, an aliquot of the edited cells edited by editing components A may be combined with editing components D, and so on for a second round of editing. After round two, an aliquot of each of the double-edited cells may be subjected to a third round of editing, where, e.g., aliquots of each of the AB-, AC-, AD-edited cells are combined with additional editing components, such as editing components X, Y, and Z. That is that double-edited cells AB may be combined with and edited by components X, Y, and Z to produce triple-edited edited cells ABX, ABY, and ABZ; double-edited cells AC may be combined with and edited by components X, Y, and Z to produce triple-edited cells ACX, ACY, and ACZ; and double-edited cells AD may be combined with and edited by components X, Y, and Z to produce triple-edited cells ADX, ADY, and ADZ, and so on. In this process, many permutations and combinations of edits can be executed, leading to very diverse cell populations and cell libraries.

In any recursive process, it is advantageous to “cure” the previous engine and editing components (or single engine+editing vector in a single vector system). “Curing” is the process in which one or more components used in the prior round of editing is eliminated from the transformed cells. Curing can be accomplished by, e.g., cleaving the vector(s) coding for the components using a curing plasmid thereby rendering the editing and/or engine components nonfunctional; diluting the components or vector(s) in the cell population via cell growth (that is, the more growth cycles the cells go through, the fewer daughter cells will retain the editing or engine vector(s)), or by, e.g., utilizing a heat-sensitive origin of replication on an editing or engine vector (or combined engine+editing vector). The conditions for curing will depend on the mechanism used for curing; that is, in this example, how the curing plasmid cleaves the editing and/or engine vector.

### EXAMPLES

The following examples are put forth so as to provide those of ordinary skill in the art with a complete disclosure and description of how to make and use the present invention, and are not intended to limit the scope of what the inventors regard as their invention, nor are they intended to represent or imply that the experiments below are all of or the only experiments performed. It will be appreciated by persons skilled in the art that numerous variations and/or modifications may be made to the invention as shown in the specific aspects without departing from the spirit or scope of the invention as broadly described. The present aspects are, therefore, to be considered in all respects as illustrative and not restrictive.

### Example I: Nuclease- or Nickase-Ku and Ligase Fusion Editing in HEK293t Cells

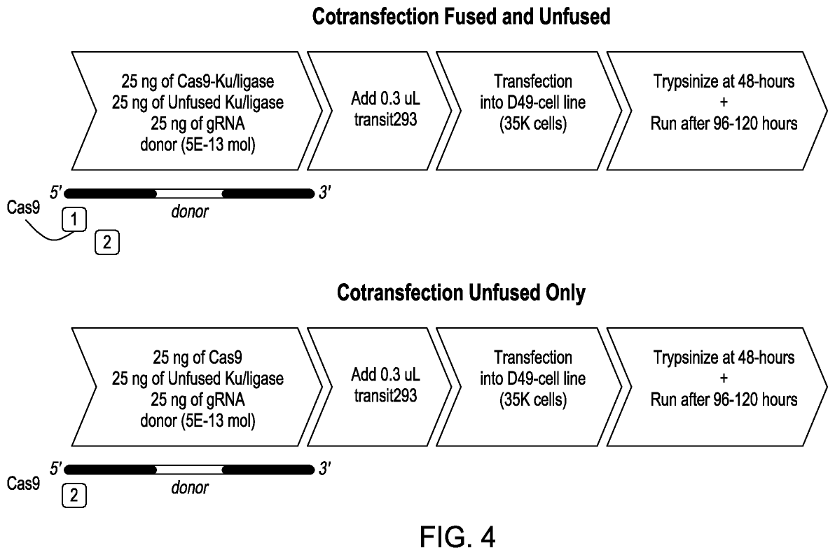

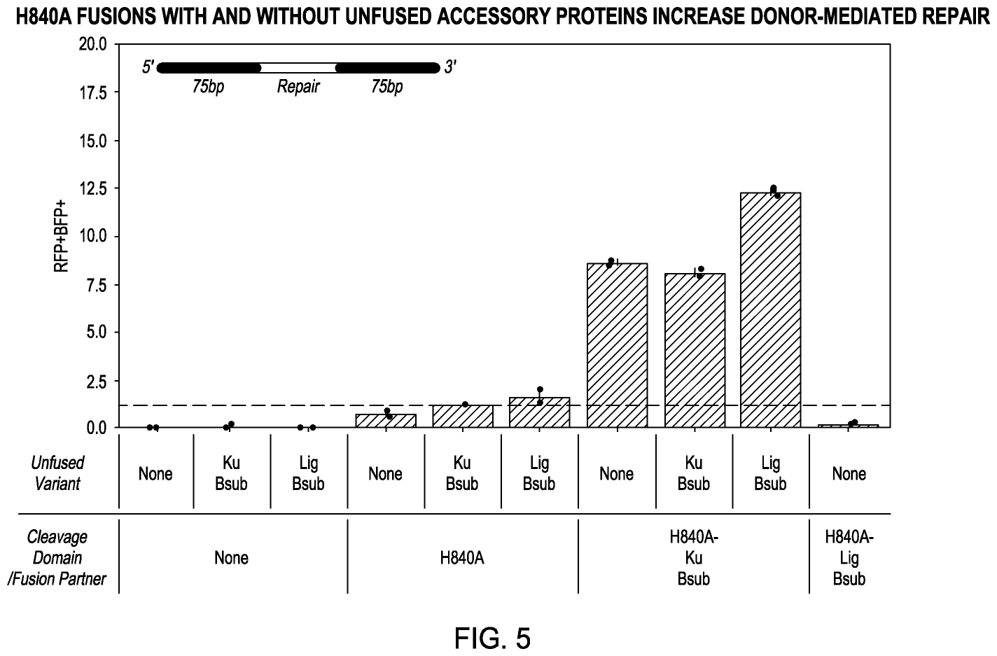

FIG. 4 is a simplified graphic showing the workflow used to test the nuclease-Ku, nuclease-ligase, nickase-Ku and nickase-ligase fusion proteins with unfused ligases or Ku proteins shown in FIGS. 5, 8 and 9. At top, 25 ng of Cas9-Ku, Cas9-ligase, Cas9 nickase-Ku or Cas9 nickase-ligase fusions were co-transfected into HEK293t cells with 25 ng of unfused Ku or ligase proteins, along with 25 ng of gRNA and 5E-13 moles repair template using 0.3 μL of transIT®-293 transfection reagent (Mirus Bio LLC, Madison, Wis., USA). A simplified graphic of the Cas9 fusion (Cas9-1, 2) is shown below in addition to the position of the repair template in the target. Cas9-1 denotes the N-terminal Cas9 nuclease or nickase fused to 1, where 1 is either a monomer of a bacterial Ku protein or a ligase and 2 denotes unfused bacterial Ku protein or ligase. At bottom, 25 ng of Cas9 nuclease or Cas9 nickase was transfected into HEK293t cells along with 25 ng of unfused bacterial Ku or ligase proteins, and 25 ng of gRNA, 5E-13 moles repair template using 0.3 μL of transIT®-293 transfection reagent (Mirus Bio LLC, Madison, Wis., USA). A simplified graphic of the unfused Cas9 fusion (Cas9, 2) is shown below. Cas9 denotes the unfused Cas9 nuclease or nickase and 2 denotes unfused bacterial Ku protein or ligase. Also shown is the position of the repair template in the target.

FIG. 5 is a bar graph showing the results from the workflow shown in FIG. 4 that the Cas9 nickase H840A fused with the B. subtilis Ku protein increases donor-templated repair in HEK293t cells. The increase appears to be higher when cotransfected with a plasmid expressing unfused LigaseD from B. subtilis. The abbreviations here are the same as in FIG. 4 and the cleavage domain/fusion partners and unfused proteins present are indicated.

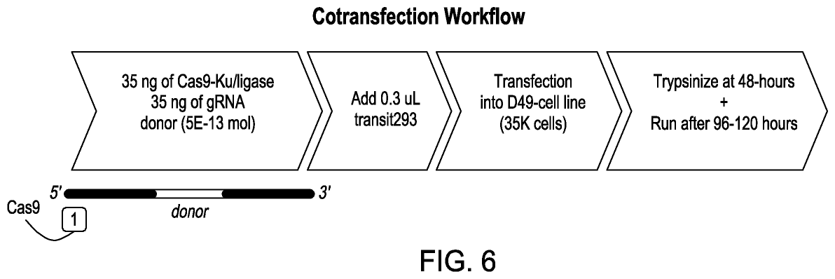

FIG. 6 is a simplified graphic showing the workflow used to test the nuclease-Ku, nuclease-ligase, nickase-Ku and nickase-ligase fusion proteins. Here, 35 ng of Cas9-Ku, Cas9-ligase, Cas9 nickase-Ku or Cas9 nickase-ligase fusions were co-transfected into HEK293t cells with 35 ng of gRNA and 5E-13 moles repair template using 0.3 μL of transIT®-293 transfection reagent (Mirus Bio LLC, Madison, Wis., USA). A simplified graphic of the Cas9 fusion (Cas9-1) is shown below in addition to the position of the repair template in the target. Cas9-1 denotes the N-terminal Cas9 nuclease or nickase fused to 1, where 1 is either a monomer of a bacterial Ku protein or a ligase and 2 denotes unfused bacterial Ku protein or ligase.

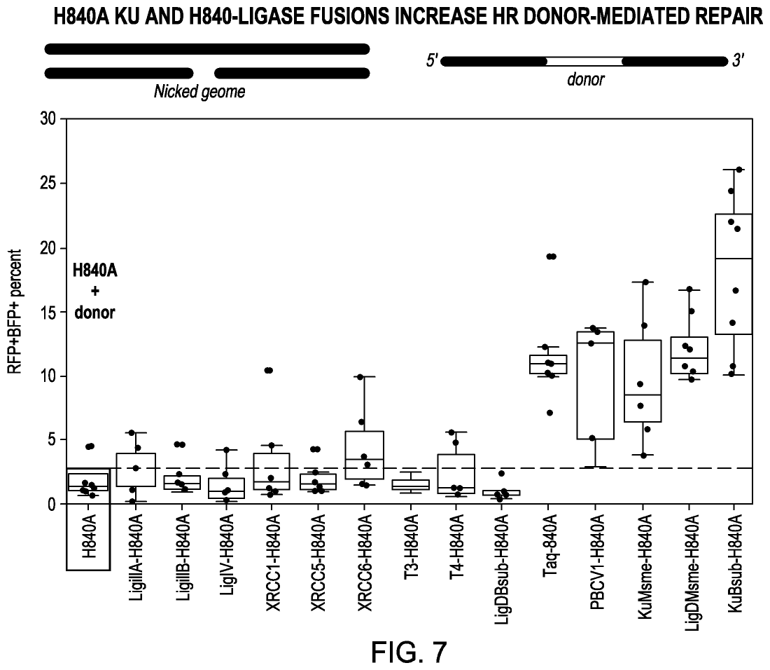

FIG. 7 is a bar graph showing the results from the workflow shown in FIG. 6 FIG. 7 is a bar graph showing that the Cas9 nickase H840A fused with a ligase selected from Taq ligase, PBCV1 ligase or ligaseD from M. smegmatis as well as Cas9 nickase H840A fused to Ku from B. subtilis and M. smegmatis increases donor mediated repair in HEK293t cells. At top, a simplified graphic of a nicked target genome is shown, along with the repair template. The X-axis recites the fusions and controls tested and the Y-axis is the percent RFP⁺ BFP⁺. Fused and unfused Cas proteins are linked via a T2A tag to dsRed; RFP is evidence of transfected cells and BFP⁺ is evidence of a proper edit. H840A is a known nickase of Cas9 where the amino acid histidine at position 840 in wildtype Cas9 has been replaced with alanine. LigIIIA-H840A, LigIIIB-H840A and LigIV-H840A are fusions where the N-terminus is the Cas9 nickase H840A that has been fused at its C-terminus to a linker, then to one of the mammalian ligases LigIIIA, LigIIIB and LigIV. XRCC1-H840A, XRCC5-H840A and XRCC6-H840A are fusions where the N-terminus is the Cas9 nickase H840A that has been fused at its C-terminus to a linker, then to one of XRCC1, XRCC5, or XRCC6, which are all proteins involved in DNA repair in mammals where each complexes with DNA ligase III. T3-H840A and T4-H840A are fusions where the N-terminus is the Cas9 nickase H840A fused at its C-terminus to a linker, then to T3 or T4 ligase. LigDBsub-H840A is a fusion where the N-terminus is the Cas9 nickase H840A fused at its C-terminus to a linker, then to ligase D of Bacillus subtilis. Taq-H840A is a fusion where the N-terminus is the Cas9 nickase H840A fused at its C-terminus to a linker, then to Taq ligase. PBCV-H840A is a fusion where the N-terminus is the Cas9 nickase H840A has been fused at its C-terminus to a linker, then to Chlorella virus PBCV-1 ligase. KuMsme-H840A is a fusion where the N-terminus is the Cas9 nickase H840A fused at its C-terminus to a linker, then to a monomer of the Ku protein of Mycobacterium smegmatis. LigDMsme-H840A is a fusion where the N-terminus is the Cas9 nickase H840A fused at its C-terminus to a linker, then to ligase D of M. smegmatis. Finally, KuBsub-H840A is a fusion where the N-terminus is the Cas9 nickase H840A fused at its C-terminus to a linker, then to a monomer of the Ku protein of B. subtilis. Note that there is a baseline editing of around 1 percent for H840A alone and the nine first fusion proteins. A small baseline level of editing is expected as there should be some amount of endogenous repair. All of the Taq ligase, PBCV1 ligase, M. smegmatis Ku, M. smegmatis ligase D and B. subtilis Ku fusions with the nickase H840A had enhanced activity from 3× to over 8× from the baseline.

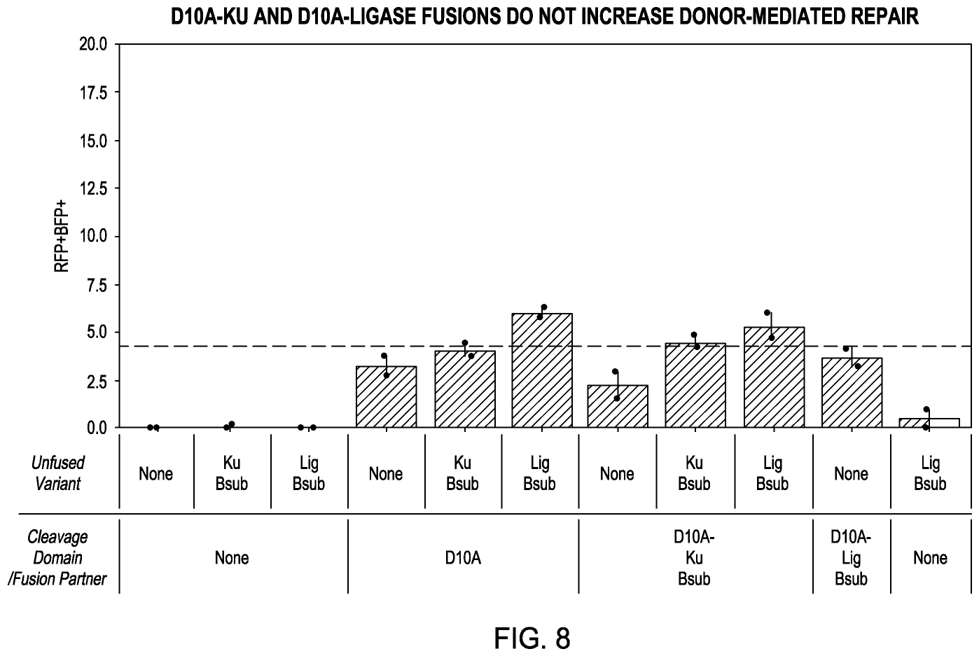

FIG. 8 is a bar graph showing the results from the workflow shown in FIG. 4 FIG. 8 is a bar graph showing that the Cas9 nickase D10A fused with a monomer of the B. subtilis Ku protein has little to no improvement in donor-templated repair in HEK293t cells over Cas9 nickase D10A alone. These constructs were transfected under the same conditions shown in FIG. 4. D10A is another known nickase of Cas9 where the amino acid aspartic acid at position 10 in wildtype Cas9 has been replaced with alanine. D10A nicks the opposite strand that the H840A nickase nicks. The cleavage domain/fusion partners and unfused proteins present are indicated.

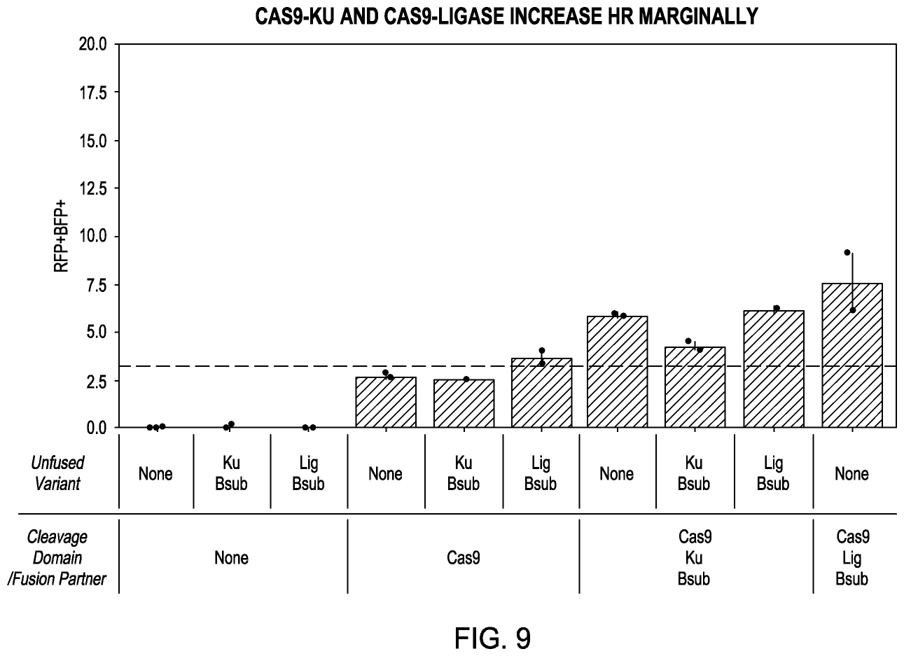

FIG. 9 is a bar graph showing the results from the workflow shown in FIG. 4 FIG. 9 is a bar graph showing that the Cas9 nuclease fused with a monomer of the B. subtilis Ku protein and the Cas9 nuclease fused to B. subtilis ligase has a modest improvement in donor-templated repair in HEK293t cells over Cas9 nuclease alone and that addition of unfused binding partners does not increase this repair.

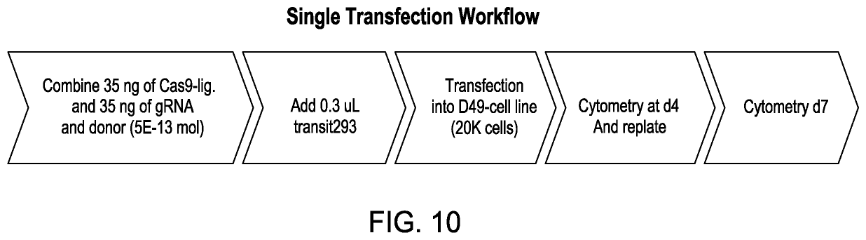

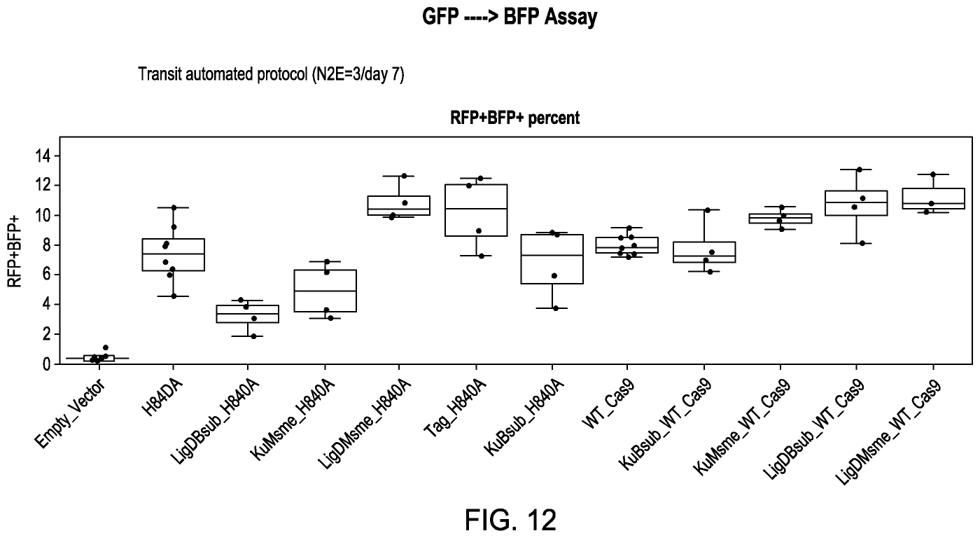

FIG. 10 is a simplified graphic showing the workflow used to test the nuclease-Ku, nuclease-ligase, nickase-Ku and nickase-ligase fusion proteins in FIGS. 12, 13, 14, and 15. At top, 35 ng of Cas9-Ku, Cas9-ligase, Cas9 nickase-Ku or Cas9 nickase-ligase fusions were co-transfected into HEK293t cells with along with 35 ng of gRNA and 5E-13 μmol of donor. DNA amounts shown were brought to 9 μL H₂O and mixed with 0.3 μL Transit293 (TransIT-293® transfection reagent, Mirus Bio, Madison Wis., USA), which was brought to 9 μL in Opti-MEM® (Thermo Fisher., Waltham, Mass., USA). The DNA and Transit293 mixtures were combined together and added to 20,000 cells engineered to contain an integrated BFP gene lacking 49 or 187 bp of its coding sequence, or those containing a full length GFP gene, in 96-well Nunc plates. Cells were analyzed at day 4 via flow cytometry using Attune Nxt™ cytometry (Thermo Fisher., Waltham, Mass., USA) with a Cytkick™ Mac (Thermo Fisher., Waltham, Mass., USA) and replated. The replated cells were then analyzed again via flow cytometry and data was analyzed using FlowJo software (BD, Ashland Oreg., USA).

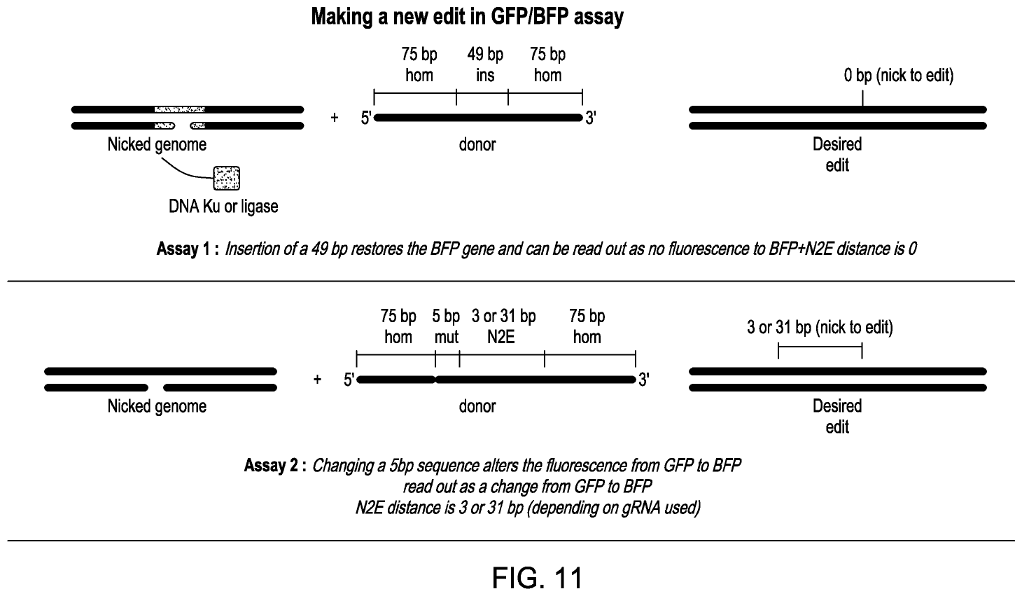

FIG. 11 illustrates the differences between the assays used to assess genomic repair. Assay 1 contains a 49 deletion in an integrated BFP gene in HEK293t cells. Restoration of this region via repair restores the open reading frame and can be read out as BFP fluorescence; this assay was used to assess editing rates in FIGS. 5,7,8 and 9. Assay 2 is a GFP→BFP conversion assay; this assay was used to assess editing rates in FIGS. 12-15. Assay 2 contains a continuously expressed GFP gene integrated in HEK293t cells. Altering a 5 bp region, via HDR or other donor-mediated repair, changes the emission spectrum of the GFP to that of BFP. Genomic breaks that result in loss of the reading frame or deleterious non-synonymous mutations can be read out as GFP-cells. In the GFP→BFP conversion assay, two separate gRNAs and donors were used to assess the donor-mediate repair at nick-to-edit-distances of 3 or 31 bases from the desired 5 bp mutation. This is in contrast to Assay 1, where gRNA-enabled cleavage at the exact location is needed for the desired 49 bp insertion. In both assays, the donor encodes a PAM immunizing mutation that, when incorporated, prevents Cas9 cleavage or nicking. In all assays, 75 bp of homology on the 5′ and 3′ of the nucleotide region flanked the desired mutation and PAM immunizing edit and nick to PAM distance.

FIG. 12 is a bar graph showing the results from the workflow shown in FIG. 10. FIG. 12 shows the results for the GFP→BFP conversion at a nick-to-edit distance of 3. Transfection positive (RFP+) BFP+ cells are shown for transfections with repair donor, gRNA, and Cas9 (WT) or Cas9 (H840A) Ku or ligase nickase fusions. LigDBsub_H840A is a fusion where the N-terminus is the Cas9 nickase H840A fused at its C-terminus to a linker, then to ligase D of Bacillus subtilis. KuMsme_H840A is a fusion where the N-terminus is the Cas9 nickase H840A fused at its C-terminus to a linker, then to a monomer of the Ku protein of Mycobacterium smegmatis. LigDMsme_H840A is a fusion where the N-terminus is the Cas9 nickase H840A fused at its C-terminus to a linker, then to ligase D of M. smegmatis. Taq_H840A is a fusion where the N-terminus is the Cas9 nickase H840A fused at its C-terminus to a linker, then to Taq ligase. KuBsub_H840A is a fusion where the N-terminus is the Cas9 nickase H840A fused at its C-terminus to a linker, then to a monomer of the Ku protein of B. subtilis. KuBsub_WT_Cas9 is a fusion where the N-terminus is the Cas9 nuclease fused at its C-terminus to a linker, then to a monomer of the Ku protein of B. subtilis. KuMsme_WT_Cas9 is a fusion where the N-terminus is the Cas9 nuclease fused at its C-terminus to a linker, then to a monomer of the Ku protein of Mycobacterium smegmatis. LigDBsub_WT_Cas9 is a fusion where the N-terminus is the Cas9 nuclease fused at its C-terminus to a linker, then to ligase D of Bacillus subtilis. LigDMsme_WT_Cas9 is a fusion where the N-terminus is the Cas9 nuclease fused at its C-terminus to a linker, then to ligase D of M. smegmatis.

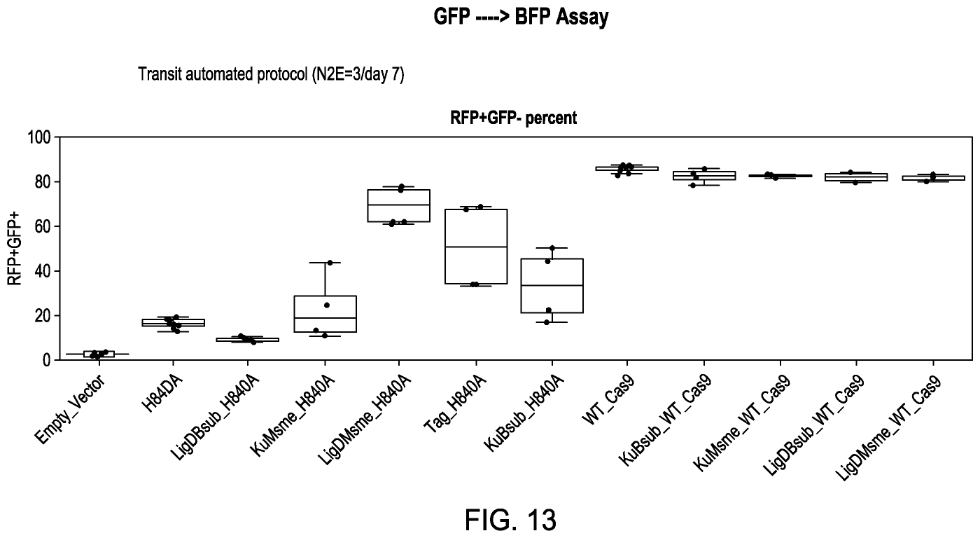

FIG. 13 is a bar graph showing the results from the workflow shown in FIG. 10. FIG. 13 indicates error-prone repair of the GFP→BFP conversion at a nick-to-edit distance of 3. Transfection positive (RFP+), GFP− cells are shown for transfections with repair donor, gRNA, and Cas9 or Cas9 (H840A) nickase fusions. The Cas9 H840A nickase fusion partners result in much higher rates of error prone repair, relative to unfused Cas9 H840A nickase. However, this is not true of Cas9 fusion partners relative to unfused, WT_Cas9. Abbreviations are the same as described in relation to FIG. 12.

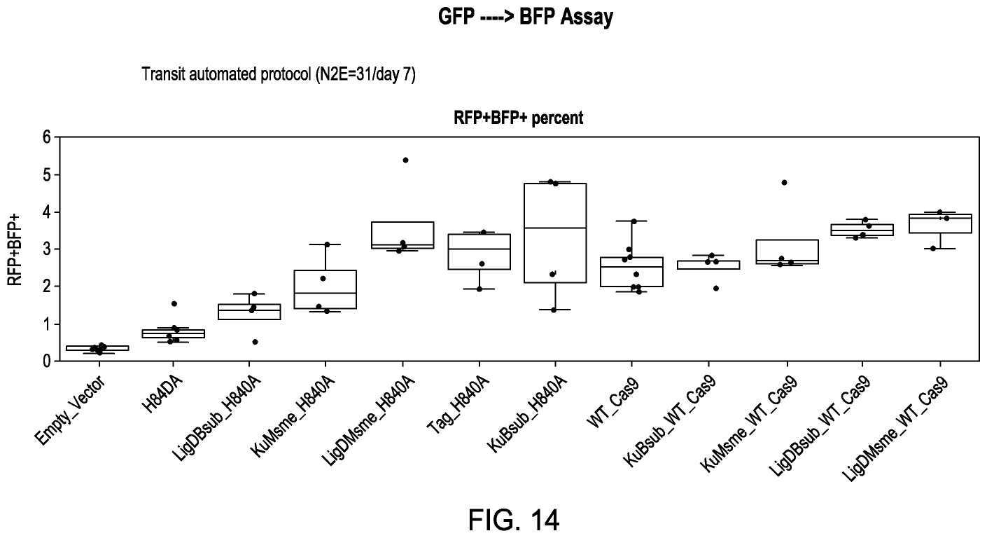

FIG. 14 is a bar graph showing the results from the workflow shown in FIG. 10. FIG. 14 shows the results for the GFP→BFP conversion at a nick-to-edit distance of 31. Transfection positive (RFP+), BFP+ cells are shown for transfections with repair donor, gRNA, and Cas9 or Cas9 (H840A) nickase fusions. Note the increase in donor-mediated repair is higher for some of the fusion partners, relative to Cas9 or Cas9 nickase alone, but absolute rate of repair is lower than the results shown in FIG. 7. Abbreviations are the same as described in relation to FIG. 12.

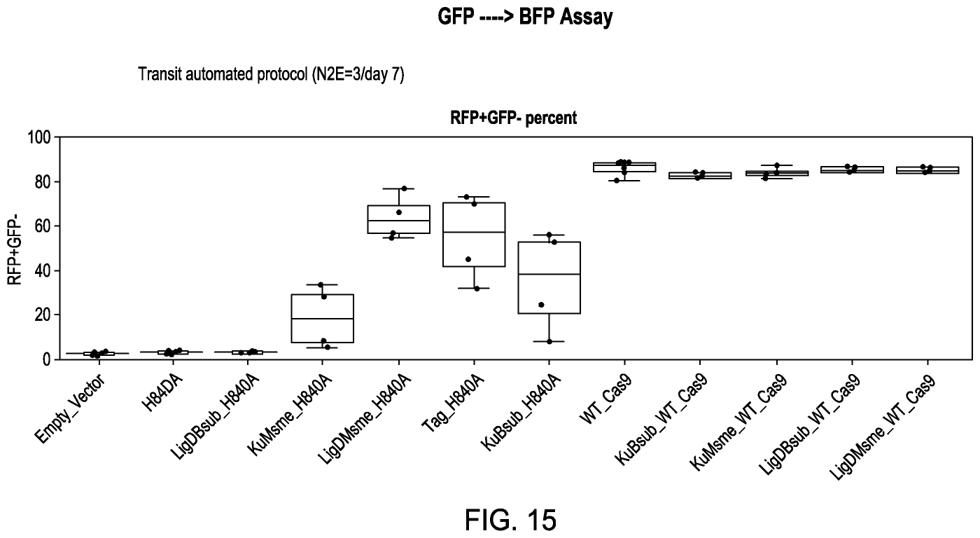

FIG. 15 is a bar graph showing the results from the workflow shown in FIG. 10. FIG. 15 indicates error-prone repair of the GFP→BFP conversion at a nick-to-edit distance of 31. Transfection positive (RFP+), GFP-cells are shown for transfections with repair donor, gRNA, and Cas9 or Cas9 (H840A) nickase fusions. The H840A nickase fusion partners result in much higher rates of error prone repair, relative to H840A. However, this is not true of Cas9 fusion partners. Abbreviations are the same as described in relation to FIG. 12.

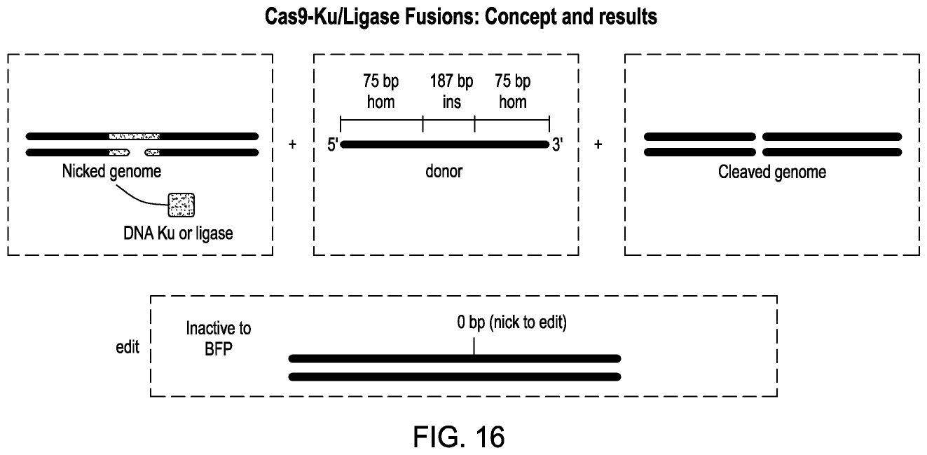

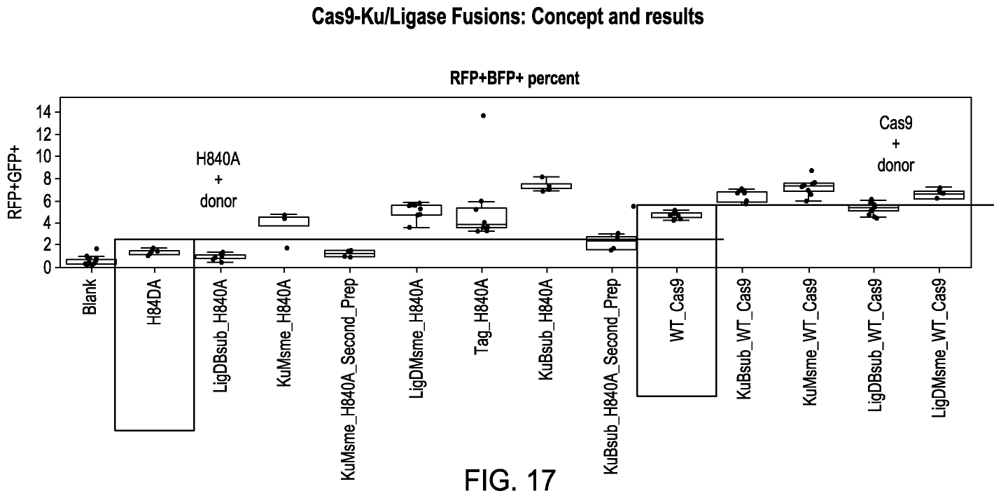

The workflow for the experiments with results reported in FIG. 17 was identical to that shown in FIG. 10 except that DNA amounts shown were brought to 9 μL H₂O and mixed with 0.9 uL Transit293 (TransIT-293® transfection reagent, Mirus Bio, Madison Wis., USA) which was brought to 9.6 μL in Opti-MEM® (Thermo Fisher., Waltham, Mass., USA). FIG. 16 illustrates how Cas9 H840A nickase or Cas9 WT fusion proteins effect donor-mediated repair using a donor that encodes for a 187 bp insert. In the assay, a single stranded homology donor introduces a 187 bp insertion into a gnomically integrated BFP, which lacks this region. The donor insertion is also flanked at its 5′ and 3′ end by 75 bp of homology to the cut/nick site. Incorporation of the region restores the reading frame, which can be read out as transfected, BFP+ cells. Note that the gRNA/Cas9 complex nicks or cleaves at the desired insertion site.

FIG. 17 is a bar graph showing the results from the workflow shown in FIG. 16. The results shown in FIG. 17 demonstrate co-transfections with a plasmid-encoding a fusion protein, a plasmid encoding a gRNA and a donor enables repair. Specific proteins fused to an H840A nickase Cas9 variant or WT_Cas9 enable upregulation of donor-mediated repair, though the absolute magnitude of the repair is not as large as the results shown in FIG. 7. An additional two DNA preparations and transfections of plasmids encoding H840A nickase fusions did not result in repair as high as an earlier preparations. Abbreviations are the same as described in relation to FIG. 12.

While this invention is satisfied by embodiments in many different forms, as described in detail in connection with preferred embodiments of the invention, it is understood that the present disclosure is to be considered as exemplary of the principles of the invention and is not intended to limit the invention to the specific embodiments illustrated and described herein. Numerous variations may be made by persons skilled in the art without departure from the spirit of the invention. The scope of the invention will be measured by the appended claims and their equivalents. The abstract and the title are not to be construed as limiting the scope of the present invention, as their purpose is to enable the appropriate authorities, as well as the general public, to quickly determine the general nature of the invention. In the claims that follow, unless the term “means” is used, none of the features or elements recited therein should be construed as means-plus-function limitations pursuant to 35 U.S.C. § 112, ¶6.

I claim:

1. A system for RNA-guided (CRISPR) editing of live mammalian cells comprising:
a. a fusion protein comprising two domains:
i. an N-terminal or C-terminal domain comprising an RNA-guided nuclease or RNA-guided nickase domain; and
ii. a C-terminal or N-terminal domain comprising a ligase or bacterial Ku protein, wherein the RNA-guided nuclease or RNA-guided nickase and ligase or bacterial Ku protein are separated by a linker; and
b. a gRNA and repair template pair.

2. The system of claim 1, wherein the RNA-guided nuclease or nickase domain is a nickase domain.

3. The system of claim 2, wherein the RNA-guided nickase is selected from a nickase engineered from MAD7 nuclease, Cas9 nuclease, Cpf1 nuclease or Cas12 nuclease.

4. The system of claim 1, wherein the RNA-guided nuclease or nickase domain is a nuclease domain.

5. The system of claim 4, wherein the RNA-guided nuclease is selected from MAD7, Cas9, Cpf1 or Cas12.

6. The system of claim 1, wherein the C-domain consists of a ligase.

7. The system of claim 6 wherein the ligase is selected from Taq ligase, PBCV1 ligase, B. subtilis Ligase D or M. smegmatis ligase D.

8. The system of claim 7, wherein the ligase is M. smegmatis ligase D.

9. The system of claim 7, wherein the ligase is Taq ligase.

10. The system of claim 7, wherein the ligase is PBCV1 ligase.

11. The system of claim 7, wherein the ligase is B. subtilis LigaseD.

12. The system of claim 1, wherein the C-domain consists of a bacterial Ku protein.

13. The system of claim 12, wherein the bacterial Ku protein monomer is a Ku protein monomer from B. subtilis or M. smegmatis.

14. The system of claim 13, wherein the bacterial Ku protein monomer is a Ku protein monomer from M. smegmatis.

15. The system of claim 13, wherein the bacterial Ku protein monomer is a Ku protein monomer from B. subtilis.

16. The system of claim 1, wherein the linker is a flexible linker.

17. The system of claim 1, wherein the linker is a rigid linker.

18. The system of claim 1, wherein the N-terminal domain comprises the RNA-guided nuclease or RNA-guided nickase and the C-terminal domain comprises the bacterial Ku protein or ligase.

19. The system of claim 1, wherein the C-terminal domain comprises the RNA-guided nuclease or RNA-guided nickase and the N-terminal domain comprises the bacterial Ku protein or ligase.

20. The system of claim 1, wherein the bacterial Ku protein is a bacterial protein monomer.

21. A method for RNA-guided (CRISPR) editing of live mammalian cells comprising:
designing a fusion protein comprising two domains:
i. an N-terminal or C-terminal domain comprising an RNA-guided nuclease or RNA-guided nickase domain; and
ii. a C-terminal or N-terminal domain comprising a ligase or bacterial Ku protein, wherein the RNA-guided nuclease or RNA-guided nickase and ligase or bacterial Ku protein are separated by a linker;
designing a library of at least two different gRNA and repair template pairs;
transforming mammalian cells with the fusion protein and library of at least two different gRNA and repair template pairs;
providing conditions to allow the mammalian cells to be edited; and
enriching for mammalian cells that have been edited.

22. The method of claim 21, wherein the enriching step is performed by fluorescence-activated cell sorting.

23. The method of claim 21, wherein the enriching step is performed by magnetic-activated cell sorting.

24. The method of claim 21, wherein the ligase is selected from Taq ligase, PBCV1 ligase, B. subtilis Ligase D or M. smegmatis ligaseD.

25. The method of claim 24, wherein the ligase is M. smegmatis ligaseD.

26. The method of claim 24, wherein the ligase is B. subtilis ligaseD.

27. The method of claim 21, wherein the C-domain consists of a bacterial Ku protein.

28. The method of claim 27, wherein the bacterial Ku protein monomer is a Ku protein monomer from M. smegmatis.

29. The method of claim 27, wherein the bacterial Ku protein monomer is a Ku protein monomer from B. subtilis.

30. A cell comprising the system of claim 1.
